(19) United States
(12) Patent Application Publication
(10) Pub. No.: US 2022/0280568 A1
(43) Pub. Date: Sep. 8, 2022

(54) DUAL BISPECIFIC ANTIBODY COMPOUNDS AND USES THEREOF

(71) Applicant: CITY OF HOPE, Duarte, CA (US)

(72) Inventors: John E. SHIVELY, Duarte, CA (US); Maciej KUJAWSKI, Duarte, CA (US); Lin LI, Duarte, CA (US); Paul J. YAZAKI, Duarte, CA (US)

(21) Appl. No.: 17/632,869

(22) PCT Filed: Aug. 13, 2020
(86) PCT No.: PCT/US20/46181

§ 371 Date: Feb. 4, 2022

Related U.S. Application Data
(60) Provisional application No. 62/887,523, filed on Aug. 15, 2019.

(51) Int. Cl. A61K 35/17; C07K 16/28; C07K 16/30; A61K 47/68; A61K 51/10

(52) U.S. Cl. CPC A61K 35/17; C07K 16/2809; C07K 16/3007; A61K 47/6853; A61K 47/6849; A61K 47/6889; A61K 51/1048; C07K 2317/31; C07K 2317/35; C07K 2317/622

(57) ABSTRACT

Provided herein, inter alia, are methods of using bispecific antibody compounds which may bind immune cells and are, inter alia, useful for treating cancers. Anti-tumor antigen antibodies are connected to anti-immune antibodies through their hinge regions using, for example, click chemistry to form dual-specific, bivalent BiTES with high in vivo tumor targeting ability and tumor cytotoxicity.

### CROSS-REFERENCES TO RELATED APPLICATIONS

This application claims priority to U.S. Application No. 62/887,523 filed Aug. 15, 2019, the disclosure of which is incorporated by reference herein in its entirety.

### BACKGROUND

Traditional bispecific T-cell engaging antibodies (BiTEs), comprised of anti-immune cell and anti-cancer protein scFv fragments connected by a linker, have emerged as therapeutic agents for the treatment of cancer. However, disordered disulfide bond formation in scFv fragments cause BiTE construct misfolding, and the small molecular weight of BiTEs lead to rapid kidney clearance, requiring frequent infusions for therapeutic effect. Additionally, BiTEs lacking Fc domains have short half-lives compared to antibodies and antibody conjugates which retain Fc domains. Futher, solution based BiTE generation has a slow rate of formation due to random orientation of the two antibody fragments in liquid, results in highly heterogeneous products, and requires additional purification steps to remove minor species and unreacted antibody. Therefore, it is important to form stable and homogenous bispecific antibody compounds, which retain directed cytotoxic properties against cancer cells. Provided herein are solutions to these and other problems in the art.

### BRIEF SUMMARY OF THE INVENTION

In an aspect, a method of treating cancer in a subject in need thereof is provided. The method includes administering to a subject a therapeutically effective amount of an autologous immune cell bound to a bispecific antibody compound, the bispecific antibody compound comprising an anti-immune cell antibody covalently bound to an anti-cancer antibody through a linker, wherein the linker is attached to a first amino acid within a first hinge region of the anti-immune cell antibody and a second amino acid within a second hinge region of the anti-cancer antibody.

In another aspect, an autologous immune cell bound to an anti-immune cell antibody is provided. The anti-immune cell antibody includes a hinge region amino acid, wherein the hinge region amino acid includes a reactive chemical group side chain.

In another aspect, the autologous immune cell bound to a bispecific antibody compound is provided. The bispecific antibody compound includes an anti-immune cell antibody covalently bound to an anti-cancer antibody through a linker, wherein the linker is attached to a first amino acid within a first hinge region of the anti-immune cell antibody and a second amino acid within a second hinge region of the anti-cancer antibody.

In another aspect is provided a pharmaceutical composition including an autologous immune cell bound to a bispecific antibody and a pharmaceutically acceptable excipient provided herein including embodiments thereof.

In another aspect a process for making an autologous immune cell bound to a bispecific antibody compound is provided. The process includes: combining an autologous immune cell and an anti-immune cell antibody in a reaction vessel thereby forming an autologous immune cell-antibody complex, wherein the autologous immune cell-antibody complex includes the autologous immune cell bound to the anti-immune cell antibody, wherein the anti-immune cell antibody includes a first hinge region amino acid and the first hinge region amino acid includes a first reactive chemical group side chain; combining the autologous immune cell-antibody complex with a second antibody in a reaction vessel thereby forming the autologous immune cell bound to the bispecific antibody compound, wherein the second antibody comprises a second hinge region amino acid and the second hinge region amino acid comprises a second reactive chemical group side chain that is reactive with the first reactive chemical group side chain; and the bispecific antibody compound comprises the anti-immune cell antibody covalently bound to the second antibody through a linker, wherein the linker is attached to the first hinge region amino acid and the second hinge region amino acid.

### BRIEF DESCRIPTION OF THE DRAWINGS

FIG. 1A. is a schematic showing cysteine hinge specific click chemistry for generation of dbBiTES. Top left is a reduced antibody functionalized with DBCO and top right is a reduced antibody functionalized with PEGn-azide. The two derivatized antibodies form a covalent bond by click chemistry when mixed at a 1:1 ratio. The cross-linking between two heavy chains is likely the favored result on the bottom left. A rare minor product is the cross-linking between two light chains shown on the bottom right.

FIG. 1B is a schematic showing solution generation and purification of dbBITES. Reduced Ab1 was alkylated at the reduced hinge cysteines with a bromoacetyl derivative of DIBCO. Ab2 was similarly alkylated with bromoacety-PEG₅-azide.

FIG. 2A is an HPLC size exclusion chromatogram. One hundred micrograms of a crude reaction mixture of DBCO-anti-CD3 antibody plus azido-PEG₅-anti-CEA antibody were injected onto a Superdex 200 (1×30 cm) column, monitored at 214 nm (solid arrow) and 280 nm (dotted arrow) and eluted at a flow rate of 0.5 mL/min in PBS. Based on calibration with authentic standards, peak 1 has a molecular mass of 300 kDa and peak 2 has a molecular mass of 150 kDa.

FIGS. 2B-C are images of gels. FIG. 2B is an image of a nonreducing gel with samples of an IgG M5A standard and samples from peaks 1 and 2 collected from SE HPLC. FIG. 2C is an image of an SDS PAGE gel with anti-CEA M5A and anti-CD3 OKT3 as standards alongside purified peak 1 dbBiTE.

FIGS. 3A-C are images of electron microscopy (EM) analysis of dbBiTES. FIG. 3A. is a representative EM of 300 kDa particles at 67,000 magnification. FIG. 3B. is a 2D average reconstruction of over one hundred 6-lobed particles. IgG lobes arbitrarily labeled as 1 to 6, the 6th lobe is assumed to be out of the plane (z-axis) of the page. FIG. 3C is a 3D reconstruction of 6-lobed particles using tilt beam EM.

FIGS. 4A-B are structural models of the dbBiTE. The two Clicked reagents, DBCO and PEG₅-azide are shown attached to cysteines in the hinge regions of two IgG1s (FIG. 4A). Structural model derived after coarse grain MD simulations showing possible Clicked derivatives between two pairs of cysteines in the heavy chain hinge of a dbBiTE (FIG. 4B).

FIGS. 5A-F show data illustrating in vitro binding of dbBiTEs to CEA positive target cells and to human T-cells and cytotoxic effects on target cells. FIG. 5A is flow analysis of anti-CEA antibody M5A and dbBiTE to CEA positive MDA-MB-231 cells. FIG. 5B is flow analysis of anti-CD3 antibody OKT3 and dbBiTE to CD3 positive human T-cells. FIG. 5C is a graph showing cytotoxicity of dbBiTE coated T-cells on cells expressing CEA (MB2310EA) or not expressing CEA (MB231) at indicated E:T ratios. Cytotoxicity was measured by an LDH release assay. Controls are uncoated or anti-CD3 antibody coated T-cells. “***” indicates p<0.001. Antibody concentrations were 1 μg/mL per 1×10⁷ cells. FIG. 5D. is a bar graph showing IFNγ secretion following 24 hrs of incubation at an E:T ratio of 10:1. Controls include uncoated T-cells or T-cells coated with anti-CD3. The assay was performed in triplicate and “**” indicates p<0.01. FIG. 5E shows flow cytometry data illustrating binding of dbBiTE to human T-cells was measured at concentrations ranging from 1 μg/mL to 1 ng/mL. Signals from left to right are: unstained control, secondary antibody, 1 ng, 10 ng, 100 ng, 500 ng, and 1000 ng dbBiTE. FIG. 5F is a graph showing cytotoxicity against MDA-MB231-CEA by T-cells coated with 1 μg/mL to 1 ng/mL dbBiTE.

FIGS. 6A-F show in vivo activity of dbBiTE coated murine T-cells against CEA targets in a CEA transgenic mouse model. FIG. 6A are optical images of CEA-Tg mice injected i.p. with mouse carcinoma cell line MC38CEA-Luc at day 9 post injection. FIG. 6B is a graph showing the number of tumor nodules in pancreas or intestine region in CEA-Tg mice treated 4× every 3 days with 10 million activated CD3 T cells with or without dbBiTE coating. FIG. 6C. is an image of peri-pancreatic tissue with tumor nodules indicated by arrows in a mouse treated with control T cells. FIG. 6D. shows flow analysis of digested tumor nodules from animals treated with control T-cells. Samples were analyzed for presence of DAPI negative live cells, CD45 positive or CEA positive cells. CD45 cells were further analyzed for presence of CD8 and CD4 markers, and CD11b positive myeloid cells were checked for Ly6G and F4/80 markers. FIG. 6E. is an image of the peri-intestine location of a mouse treated with dbBiTE coated T cells with tumor nodules indicated by arrows. FIG. 6F shows flow analysis of tumor nodules shown in FIG. 6E using same staining and gating as in FIG. 6D.

FIGS. 7A-D show ESI MS analysis of DBCO and PEG5 azido derivatized antibodies. Anti-CD3 antibody OKT3 before (FIG. 7A) and after (FIG. 7B) derivatization with a bromoacetamido DBCO. Anti-CEA antibody M5A before (FIG. 7C) and after (FIG. 7D) derivatization with bromoacetamido-PEG₅-azide.

FIGS. 8A-E are models made from the molecular dynamic simulation of dbBiTES generation. The x-ray structures of two IgG1 antibodies were aligned and one rotated 90° to allow them to approach each other (FIG. 8A). Each antibody was converted to a coarse grain model in which each amino acid is converted to a sphere to simplify computational analysis. A section of one antibody is shown to indicated the principle (FIG. 8B). A complete coarse grain model of a single IgG1 (FIG. 8C). Alpha carbon distances (in angstroms) in the hinge region as the two coarse grain models are docked (FIG. 8D). The final model in which at least two cysteines in the hinge regions have been docked (FIG. 8E).

FIGS. 9A-C show superposition of a dbBiTE model onto an average EM particle. FIG. 9A is an atomic structure constrained to average EM particle. FIG. 9B is a space filling model of a dbBiTE constrained to an average EM particle. FIG. 9C is a stick drawing fitting to Ab to EM.

FIG. 10 shows images of dbBiTE coated T-cells killing target cells. MDA-MB231±CEA cells were treated with dbBiTE coated activated human T-cells at an E:T ratio of 10:1 for 24 hrs and visualized by microscopy.

FIGS. 11A-C show SEC analyses of ⁶⁴Cu-DOTA labeled M5A and dbBiTE. FIG. 11A is an SEC chromatogram of radiolabeled M5A. FIG. 11B is an SEC chromatogram of radiolabeled dbBiTE. FIG. 11C is an SEC chromatogram of radiolabeled dbBiTE after the addition of a 20 fold excess of CEA. Radiolabeled dbBiTE (300 kDa) contains about 23% of 150 kDa material, or 11.5% on a molar basis.

FIGS. 12A-B show images of NOD-SCID mice bearing CEA positive LS-174T tumors treated with ⁶⁴Cu-DOTA-dbBiTE. FIG. 12A shows two mice imaged at 4, 20, and 44 hr. Organs labeled are heart (H), liver (L) and tumor (T). FIG. 12B shows biodistribution of indicated tissues at terminal imaging time point.

FIGS. 13A-D show binding of dbBiTE rat-anti-murine CD3-human anti-CEA (M5A) to murine target cells±CEA and cytotoxicity.

FIG. 14. is flow cytometry data detecting Alexa-647 goat-anti-human IgG bound to hOKT3-DBCO coated human T-cells. The left peak is the Alexa-647 conjugated goat anti-human IgG only control. The right peak is hOKT-3-DBCO with Alexa-647 conjugated goat-anti-human IgG.

FIG. 15 is flow cytometry data detecting Alexa-488 M5A-azide following conjugation to hOKT3-DBCO pre-coated on T-cells. Panels from left to right correspond to results for T-cells coated with hOKT3-DBCO and reacted with: 0 μg/mL Alexa-488 M5A-azide, 5 μg/mL of A488-M5A-azide, 10 μg/mL of A488-M5A-azide, and 20 μg/mL of A488-M5A-azide.

FIG. 16. is a graph showing activated T-cells coated with cell surface clicked dbBiTEs kill target cells.

FIG. 17. is a graph showing activated T-cells coated with cell surface clicked dbBiTEs do not kill CEA negative cells.

FIG. 18. is a bar graph showing release of IFNγ by activated human T-cells coated with cell surface clicked dbBiTEs when incubated with CEA positive target cells. Black bars correspond to results for E:T ratios of 10:1 and gray bars correspond to results for E:T ratios of 5:1. The legend for the bars is as followed. Uncoated: control cells with no hOKT3-Dibco; hOKT3-Dibco: hOKT3-Dibco coated cells; dbBiTE: cells coated with 1 μg/mL solution formed dbBiTE; Click 20 ug: hOKT3-Dibco coated cells reacted with 20 ug/mL of M5A-azide. Click 10 ug: hOKT3-Dibco coated cells reacted with 10 ug/mL of M5A-azide. Click 20 ug: hOKT3-Dibco coated cells reacted with 5 ug/mL of M5A-azide.

FIG. 19. is a bar graph showing release of IFNγ by activated human T-cells coated with cell surface clicked dbBiTEs when incubated with CEA negative target cells. Black bars correspond to results for E:T ratios of 10:1 and gray bars correspond to results for E:T ratios of 5:1. The legend for the bars is as followed. Uncoated: control cells with no hOKT3-Dibco; hOKT3-Dibco: hOKT3-Dibco coated cells; dbBiTE: cells coated with 1 μg/mL solution formed dbBiTE; Click 20 ug: hOKT3-Dibco coated cells reacted with 20 ug/mL of M5A-azide. Click 10 ug: hOKT3-Dibco coated cells reacted with 10 ug/mL of M5A-azide. Click 20 ug: hOKT3-Dibco coated cells reacted with 5 ug/mL of M5A-azide.

FIG. 20 is a schematic showing dbBiTE formation in solution.

FIG. 21 is a schematic showing dbBiTE formation on the surface of a cell.

FIG. 22 is a schematic showing dbBiTE multiplexing of effector cell specific antibodies that can be clicked to a target cell specific antibody.

FIG. 23 is an image of a rectal tumor as taken by ImmunoPET with Cu-64DOTA MSA. The PET image shows a large rectal tumor and perirectal lymph node, not seen by standard FDG imaging.

FIG. 24 is a representative image of TAG72 positive tumor taken by ImmunoPET imaging. Cu-64-DOTA/huCC49 (20 μg, 100 μCI) was administered IV to nude mice bearing SC LS174T xenografts 10 days after tumor initiation. A 40 h image is shown: specific uptake was in tumor (T) and axillary lymph nodes (LN, both sides) with blood pool in the heart (H) and liver (L). Terminal biodistribution studies performed on these mice revealed tumor uptake in the range of 70-80% ID/g for 100-200 mg tumors.

FIGS. 25A-D illustrate TAG72-CAR T cells selectively kill, produce cytokines, and proliferate against TAG72-positive ovarian cancers. FIG. 25A illustrates flow cyometric analysis of TAG72 expression in human cancer cell lines, including ovarian. FIG. 25B is a bar graph showing results from a tumor killing assay assessed by flow cytometry following a 72-hour co-culture assays with indicated tumor targets and TAG72-BBz CAR T cells. FIG. 25C are bar graphs showing IFNγ (left) and IL-2 (right) production quantified by ELISA in supernatants from TAG72-BBz CAR T cells cultured for 24 hours with indicated tumor targets. FIG. 25D is a bar graph showing T cell expansion (fold) of TAG72-BBz CAR T cells following 24- or 72-hour co-culture with indicated tumor targets, determined by flow cytometry. FIGS. 25A-D adapted from Priceman et al., Front. Immunol., 19 Nov. 2018

FIGS. 26A-B illustrate TAG72-CAR T cells demonstrate therapeutic efficacy against ovarian cancers in vivo. FIG. 26A is flow cytometric analysis of TAG72 expression in OVCAR3 cells freshly isolated from ascites tumors in mice. FIG. 26B are graphs illustrating tumor flux quantification (top) and Kaplan-Meier survival (bottom) of mice treated either with intravenous (i.v.) or intraperitoneal (i.p.) administration of Mock (untransduced) or TAG72-BBz CAR T cells (5×10⁶). FIGS. 26A-B adapted from Priceman et al., Front. Immunol., 19 Nov. 2018.

FIG. 27 are flow cytometry analyses showing expression of GFP marker for CEA CAR T cells. Top panels show results corresponding to control cells activated with anti-CD3. Center panels show results corresponding to cells transfected with empty MSCV show high expression of GFP. Bottom panels show results corresponding to cells transfected with MSCV containing CEA CAR show high GFP expression. FIG. 27 adapted from Priceman et al., Front. Immunol., 19 Nov. 2018.

FIG. 28 is a graph showing CEA CAR-T cells kill CEA+target cells. Murine colon carcinoma MC38/CEA cells transfected with luciferase were co-cultured with mock CAR-T cells (circular marker) or CEA CAR-T cells (square marker) for 24 hr at indicated E;T ratios and luciferase release measure in triplicate.

FIG. 29 illustrates flow analysis of activated human PBMC or LS174T cells coated with dbBITE. Human PBMCs were activated for 3 days with anti-CD3 plus IL2 (100 U/mL) or LS174T cells coated with 1 ug/mL of dbBITE followed by secondary antibodies (anti-mouse IgG-A555 and anti-human IgG-A647). Two panels on left correspond to data for secondary antibody controls, no dbBITES. Two panels on right correspond to data for dbBITES plus secondary antibodies.

FIGS. 30A-B illustrate results for cytotoxicity assay for dbBITE coated human PBMCs (effectors) against human LS174T cells (targets). FIG. 30A is a graph showing results for cytotoxicity measured at 18 hrs by an LDH release assay. 10M Human PBMCs coated with 1 ug/mL of dbBITE were incubated with target cells at indicated E:T ratios. FIG. 30B is a bar graph showing IFNγ secretion measured in the media at an E:T of 10:1. Controls included uncoated PBMCs or PBMCs coated with OKT3. The assay was performed in triplicate, “****” indicates p<0.001.

FIG. 31 are flow cytometry analyses showing infiltration of T-cells in tumor nodules. NOD/SCID mice bearing I.P. LS174T tumors were treated 4× (every 3 days) with 1×10⁶ dbBITE coated human PBMCs. After 2 weeks tumor nodules were removed and analyzed for activated T cells by intracellular IFNγ staining.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Definitions

The abbreviations used herein have their conventional meaning within the chemical and biological arts. The chemical structures and formulae set forth herein are constructed according to the standard rules of chemical valency known in the chemical arts.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “azocinylene” by itself or as part of another substituent, means, unless otherwise stated, a divalent radical derived from an eight-membered ring with an N heteroatom, as exemplified, but not limited by, C₇H₇N. In embodiments, the azocinylene includes an aryl. In embodiments, the azocinylene includes a triazole.

The term “heteroalkyl,” by itself or in combination with another term, means, unless otherwise stated, a stable non-cyclic straight or branched chain, or combinations thereof, including at least one carbon atom and at least one heteroatom (e.g. O, N, P, Si or S) and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂N(R)(′R″—NRSO₂R′), —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, NR″C(O)₂R′, NRC(NR′R″)═NR′″, S(O)R′, —S(O)₂R′, —S(O)₂N(R′)(R″, —NRSO₂R′), —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{3A}-substituted or unsubstituted alkyl, a plurality of R^{3A} substituents may be attached to the alkyl moiety wherein each R^{3A} substituent is optionally different. Where an R-substituted moiety is substituted with a plurality of R substituents, each of the R-substituents may be differentiated herein using a prime symbol (′) such as R′, R″, etc. For example, where a moiety is R^{3A}-substituted or unsubstituted alkyl, and the moiety is substituted with a plurality of R^{3A} substituents, the plurality of R^{3A} substituents may be differentiated as R^{3A}′, R^{3A}″, R^{3A}′″, etc. In some embodiments, the plurality of R^{3A} substituents is 3. In some embodiments, the plurality of R^{3A} substituents is 2.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where variables s and are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, —N3, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
   (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

The term “reactive chemical group side chain” as used herein refers to a chemical moiety (e.g., a first reactive chemical group side chain) covalently bonded to an amino acid alpha carbon atom (e.g., an alpha carbon atom of a peptide residue or protein residue) and including a reactive functional group. In embodiments, the reactive functional group is covalently bonded to the remnant of a natural amino acid side chain resulting from the reaction of a natural amino acid side chain with a compound having the reactive functional group. In embodiments, the reactive chemical group side chain includes a reactive functional group wherein the reactive functional group is a bioconjugate reactive group capable of interacting (e.g., covalently) with a complementary bioconjugate reactive group (e.g., complementary reactive chemical group side chain). In embodiments, the reactive functional group within the reactive chemical group side chain is a click chemistry reactive functional group. In embodiments, the reactive functional group within the reactive chemical group side chain is a bioconjugate reactive group capable of interacting (e.g., covalently) with a complementary bioconjugate reactive group (e.g., complementary reactive chemical group side chain). In embodiments, the reactive functional group within the reactive chemical group side chain includes a click chemistry reactive functional group. In embodiments, the reactive functional group within the reactive chemical group side chain is capable of covalently bonding with a second chemical moiety (e.g., complementary reactive chemical group side chain).

The term “reactive functional group” as used herein refers to a chemical moiety (e.g., a first reactive functional group), which is capable of interacting (e.g., forming a bond with, covalently or non-covalently) with a second, optionally different, chemical moiety (e.g., a second reactive functional group, a complementary reactive functional group). In embodiments, the reactive functional group is a chemical moiety capable of reacting with a second reactive (e.g. a complememtnary reactive functional group) to form a covalent attachment (e.g. a linker or bond). In embodiments, the reactive functional group is a bioconjugate reactive group capable of interacting (e.g., covalently) with a complementary bioconjugate reactive group (e.g., complementary reactive functional group). In embodiments, a reactive functional group is a click chemistry reactive functional group. In embodiments, the reactive functional group is capable of covalently interacting with a second chemical moiety (e.g., complementary reactive functional group). In embodiments, the reactive functional group is capable of non-covalently interacting with a second chemical moiety (e.g., complementary reactive functional group). Non-limiting examples of a reactive functional group include biotin, azide, trans-cyclooctene (TCO) (Melissa L, et al. J. Am. Chem. Soc., 2008, 130, 13518-13519; Marjoke F, et al. Org. Biomol. Chem., 2013, 11, 6439-6455) and phenyl boric acid (PBA) (Bergseid M, et al. BioTechniques, 2000, 29, 1126-1133). In embodiments, a reactive functional group (e.g., biotin moiety) interacts non-covalently with a complementary reactive functional group (e.g., streptavidin moiety). In embodiments, a reactive functional group (e.g., azide moiety, trans-cyclooctene (TCO) moiety, phenyl boric acid (PBA) moiety) covalently binds a complementary reactive functional group (e.g., dibenzocyclooctyne (DBCO) moiety (Jewett J C and Bertozzi C R J. Am. Chem. Soc., 2010, 132, 3688-3690), tetrazine (TZ) moiety, salicylhydroxamic acid (SHA) moiety).

As used herein, the term “conjugate” refers to the association between atoms or molecules. In embodiments, a conjugate is a bioconjugate.

As used herein, the term “bioconjugate reactive moiety” and “bioconjugate reactive group” refers to a moiety or group capable of forming a bioconjugate (e.g., covalent linker or bond) as a result of the association between atoms or molecules of two bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

The reactive functional groups and bioconjugate reactive groups can be chosen such that they do not participate in, or interfere with, the chemical stability of the antigen binding domain and the peptide compound described herein.

Useful reactive moieties or functional groups (e.g. used for bioconjugate chemistries including “click chemistries” as known in the art) herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry or other click chemistry complementary reactive groups;
(o) biotin conjugate can react with avidin or strepavidin to form a avidin-biotin complex or streptavidin-biotin complex; and
(p) sulfones, for example, vinyl sulfone.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group. In embodiments, the bioconjugate reactive group is a click chemistry reactive functional group.

A click chemistry reactive functional group is a chemical group capable of reacting (e.g., covalently) with a complementary click chemistry reactive functional group in a “click chemistry” reaction. “Click chemistry” is used in accordance with its well understood meaning in Chemistry and refers generally to reactions that are high yielding, wide in scope, create only byproducts that can be removed without chromatography, are stereospecific, simple to perform, and can be conducted in easily removable or benign solvents. Several examples of reactions have been identified that fulfill these criteria, thermodynamically-favored reactions that lead specifically to one product, such as for example, nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stöckmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups can be chosen such that they do not participate in, or interfere with, the chemical stability of the antigen binding domain and the peptide compound described herein.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The symbol “ [symbol] ” denotes the point of attachment of a chemical moiety to the remainder of a molecule or chemical formula.

Descriptions of compounds (e.g., peptide compounds, antibodies, antibody-peptide complexes, chemical compounds) of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As used herein, the term “cancer protein” refers to a protein on the surface of or within a cancer cell. In embodiments, a cancer protein is a protein on the surface of a cancer cell. In embodiments, a cancer protein allows isolation of a cancer cell. In embodiments, a cancer protein allows targeting of a cancer cell. For example, a cancer protein may be targeted by an anti-cancer antibody.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using a sequence comparison algorithms or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., at least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. In certain embodiments relating to two sequences of different lengths, the comparison window includes the entire length of the shorter of the two sequences. In some embodiments relating to two sequences of different lengths, the comparison window includes the entire length of the longer of the two sequences.

Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Preferred examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In certain embodiments, the NCBI BLASTN or BLASTP program is used to align sequences. In certain embodiments, the BLASTN or BLASTP program uses the defaults used by NCBI. In certain embodiments, the BLASTN program (for nucleotide sequences) uses as defaults: a word size (W) of 28; an expectation threshold (E) or 10; max matches in a query range set to 0; match/mismatch scores of 1, −2; linear gap costs; the filter for low complexity regions used; and mask for lookup table only used. In certain embodiments, the BLASTP program (for amino acid sequences) uses as defaults a word size (W) of 3; an expectation threshold (E) of 10; max matches in a query range set to 0; the BLOSUM62 matrix (see Henikoff and Henikoff 1992) Proc. Natl. Acad. Sci. USA 89:10915); gap costs of existence: 11 and extension: 1; and conditional compositional score matrix adjustment.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

“Anti-cancer antibody” refers to an antibody that binds to a cancer protein. In embodiments, the anti-cancer antibody binds to a cancer protein on the surface of a cancer cell.

“Anti-immune cell antibody” refers to an antibody that binds to an immune cell protein. In embodiments, the immune cell protein is a protein on the surface of an immune cell. In embodiments, the immune cell protein is a receptor on the surface of an immune cell.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

The Fc (i.e. fragment crystallizable region) is used herein according to its plain and ordinary meaning in the art and refers to the “base” or “tail” of an immunoglobulin and is typically composed of two heavy chains that contribute two or three constant domains depending on the class of the antibody. For example, in IgG, IgA and IgD antibody isotypes, the Fc region may be composed of two identical protein fragments, derived from the second and third constant domains of the antibody's two heavy chains. IgM and IgE Fc regions may contain three heavy chain constant domains (CH domains 2-4) in each polypeptide chain. In embodiments, the Fc region ensures that each antibody generates an appropriate immune response for a given antigen by binding to specific proteins. In embodiments, the Fc region binds to various cell receptors, such as Fc receptors, and other immune molecules, such as complement proteins.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FRI, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL.

The CDRs of the variable region of the light chain are further referred to herein as LCDR1, LCDR2 and LCDR3, respectively, wherein LCDR1 corresponds to CDR 1 of VL, LCDR 2 corresponds to CDR 2 of VL and LCDR 3 corresponds to CDR 3 of VL. Likewise, the CDRs of the variable region of the heavy chain are further referred to herein as HCDR1, HCDR2 and HCDR3, respectively, wherein HCDR1 corresponds to CDR 1 of VH, HCDR 2 corresponds to CDR 2 of VH and HCDR 3 corresponds to CDR 3 of VH.

In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of the variable region of the heavy chain are further referred to herein as HFR1, HFR2, HFR3 and HFR4, respectively, wherein HFR1 corresponds to FR 1 of VH, HFR 2 corresponds to FR 2 of VH, HFR 3 corresponds to FR 3 of VH and HFR 4 corresponds to FR 4 of VH.

Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL. Likewise, the FRs of the variable region of the light chain are further referred to herein as LFR1, LFR2, LFR3 and LFR4, respectively, wherein LFR1 corresponds to FR 1 of VL, LFR 2 corresponds to FR 2 of VL, LFR 3 corresponds to FR 3 of VL and LFR 4 corresponds to FR 4 of VL.

The term “antigen” as provided herein refers to molecules capable of binding to the antibody binding site, wherein the binding site is not a non-CDR peptide binding region.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The “hinge” or “hinge region” refers to a flexible amino acid stretch between the C_{H}1 and C_{H}2 heavy chains of an antibody, which typically links the two chains by disulfide bonds. The hinge may usually be rich in cysteine and proline amino acid residues. In embodiments, the disulfide bonds in the hinge may be reduced by a reducing agents, for example tris(2-carboxyethyl)phosphine (TCEP) or Dithiothreitol (DTT). In embodiments, the reduced cysteine residues in the hinge may be conjugated to a chemical moiety including a click chemistry reactive functional group. For example, the chemical moiety may be bromoacetamido-dibenzoazacyclooctyne (DBCO) or bromoacetamido-(ethylene glycol).-amido-DBCO. In embodiments, the reduced cysteine may be apart of a reactive chemical group side chain.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

The term “dual specific bivalent T-cell engager” is used interchangeably with “dbBITE,” “dbBiTe,” “bispecific antibody compound” or “dual specific bivalent BiTE”.

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation” CARs include those that provide both co-stimulation (e.g., CD28 or CD137) and activation (CD3ζ). “Third-generation” CARs include those that provide multiple co-stimulation (e.g. CD28 and CD137) and activation (CD3ζ). A fourth generation of CARs have been described, CAR T cells redirected for cytokine killing (TRUCKS) where the vector containing the CAR construct possesses a cytokine cassette. When the CAR is ligated, the CAR T cell deposits a pro-inflammatory cytokine into the tumor lesion. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.

A CAR T cell is used herein according to its plain and ordinary meaning in the art and refers to a T cell that expresses a chimeric antigen receptor. CAR T cells may be produced using methods well known in the art. For example, T cells isolated from a subject (e.g. patient) may be transduced with a nucleic acid encoding a CAR. Following transduction, the cells may be activated. Once activated, the cells may be induced to develop into specialized cellular subtypes (e.g. cytotoxic T cells or regulatory T cells) by treating with, for example, specific mixtures of cytokines. Finally, the CAR T cell population may be expanded using techniques well known in the art.

An “antibody variant” as provided herein refers to a polypeptide capable of binding to an antigen and including one or more structural domains of an antibody or fragment thereof.

Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

A “single domain antibody” as provided herein refers to an antibody fragment including a single monomeric variable antibody domain. Like a whole antibody, a single domain antibody is able to bind selectively to a specific antigen. The molecular weight of a single domain antibody is 12-15 kDa, single domain antibody. In embodiments, a single domain antibody is a variable heavy chain domain. In embodiments, a single domain antibody is a variable light chain domain. Non-limiting examples of single domain antibodies include camelid-derived VHH fragments and VNAR (variable immunoglobulin new antigen receptor) fragments. In embodiments, the single-domain antibody is a peptide domain of about 110 amino acids. In embodiments, the single-domain antibody includes a variable heavy chain domain. In embodiments, the single-domain antibody includes a variable light chain domain.

A “therapeutic antibody” as provided herein refers to any antibody or functional fragment thereof that is used to treat cancer, autoimmune diseases, transplant rejection, cardiovascular disease or other diseases or conditions such as those described herein. Non-limiting examples of therapeutic antibodies include murine antibodies, murinized or humanized chimera antibodies or human antibodies including, but not limited to, Erbitux (cetuximab), ReoPro (abciximab), Simulect (basiliximab), Remicade (infliximab); Orthoclone OKT3 (muromonab-CD3); Rituxan (rituximab), Bexxar (tositumomab) Humira (adalimumab), Campath (alemtuzumab), Simulect (basiliximab), Avastin (bevacizumab), Cimzia (certolizumab pegol), Zenapax (daclizumab), Soliris (eculizumab), Raptiva (efalizumab), Mylotarg (gemtuzumab), Zevalin (ibritumomab tiuxetan), Tysabri (natalizumab), Xolair (omalizumab), Synagis (palivizumab), Vectibix (panitumumab), Lucentis (ranibizumab), and Herceptin (trastuzumab)

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

A “therapeutic agent” or “therapeutic moiety” as referred to herein, is a composition (e.g., small molecule, peptide, nucleic acid, protein, fragment) useful in treating or preventing a disease.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

“CD3” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex. In embodiments, the CD3 protein is substantially identical to the protein identified by the UniProt reference number P04234 or a variant or homolog having substantial identity thereto. In embodiments, the CD3 protein is substantially identical to the protein identified by the UniProt reference number P09693 or a variant or homolog having substantial identity thereto. In embodiments, the CD3 protein is substantially identical to the protein identified by the UniProt reference number P07766 or a variant or homolog having substantial identity thereto.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the NCBI reference number GI: 110002567 or a variant or homolog having substantial identity thereto. In embodiments, the EGFR protein is substantially identical to the protein identified by the NCBI reference number GI: 2811086 or a variant or homolog having substantial identity thereto. In embodiments, the EGFR protein is substantially identical to the protein identified by the NCBI reference number GI: 63101670 or a variant or homolog having substantial identity thereto.

The term “Her2” as referred to herein includes any of the recombinant or naturally-occurring forms of the human epidermal growth factor receptor 2 protein, also known as receptor tyrosine-protein kinase erbB-2, or variants or homologs thereof that maintain Her2 activity (e.g. within at least 50%, 80%, 90%, 95%, 96%, 97%, 98%, 99% or 100% activity compared to Her2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Her2 protein. In embodiments, the Her2 protein is substantially identical to the protein identified by the NCBI reference number GI: 119533 or a variant or homolog having substantial identity thereto. In embodiments, the Her2 protein is substantially identical to the protein identified by the NCBI reference number GI: 54792096 or a variant or homolog having substantial identity thereto. In embodiments, the Her2 protein is substantially identical to the protein identified by the NCBI reference number GI: 584277106 or a variant or homolog having substantial identity thereto.

“CD20” as referred to herein includes any of the recombinant or naturally-occurring forms of CD20, also known as B-lymphocyte antigen CD20, or variants or homologs thereof that maintain CD20 activity (e.g. within at least 50%, 80%, 90%, 95%, 96%, 97%, 98%, 99% or 100% activity compared to CD20). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD20 protein. In embodiments, the CD20 protein is substantially identical to the protein identified by the NCBI reference number GI: 23110987 or a variant or homolog having substantial identity thereto. In embodiments, the CD20 protein is substantially identical to the protein identified by the NCBI reference number GI: 23110989 or a variant or homolog having substantial identity thereto. In embodiments, the CD20 protein is substantially identical to the protein identified by the NCBI reference number GI: 115968 or a variant or homolog having substantial identity thereto.

“Tumor-associated glycoprotein 72” (TAG-72) as referred to herein includes any of the recombinant or naturally-occurring forms of TAG-72, or variants or homologs thereof that maintain TAG-72 activity (e.g. within at least 50%, 80%, 90%, 95%, 96%, 97%, 98%, 99% or 100% activity compared to TAG-72). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TAG-72 protein. In embodiments, the TAG-72 protein is substantially identical to the protein in Sheer et al., Cancer Research 48, 6811-6818 (1988), Johnson et al., Cancer Res. 46(2):850-857 (1986), Ponnusamy et al., Cancer Lett. 28; 251(2):247-257 (2007), or Kostakoglu, L. Cancer Invest. 12(6):551-558 (1994).

“Carcinoembryonic antigen” (CEA) as referred to herein describes a set of highly related glycoproteins involved in cell adhesion and includes any of the recombinant or naturally-occurring forms of CEA variants or homologs thereof that maintain CEA activity (e.g. within at least 50%, 80%, 90%, 95%, 96%, 97%, 98%, 99% or 100% activity compared to CEA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to naturally occurring CEA proteins. In embodiments, the CEA protein is substantially identical to the protein protein identified by the NCBI reference number GI: 180199 or a variant or homolog having substantial identity thereto. In embodiments, the CEA protein is substantially identical to the protein protein identified by the NCBI reference number GI: 180211 or a variant or homolog having substantial identity thereto. In embodiments, the CEA protein is substantially identical to the protein protein identified by the NCBI reference number GI: 178677 or a variant or homolog having substantial identity thereto.

A “IFNγ” or “IFNγ protein” as referred to herein includes any of the recombinant or naturally-occurring forms of Interferon gamma (IFNγ), or variants or homologs thereof that maintain IFNγ activity (e.g. within at least 50%, 80%, 90%, 95%, 96%, 97%, 98%, 99% or 100% activity compared to IFNγ). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IFNγ polypeptide. In embodiments, the IFNγ protein is substantially identical to the protein identified by the NCBI reference number GI: 184639 or a variant or homolog having substantial identity thereto. In embodiments, the IFNγ protein is substantially identical to the protein identified by the NCBI reference number GI: 20805896 or a variant or homolog having substantial identity thereto. In embodiments, the IFNγ protein is substantially identical to the protein identified by the NCBI reference number GI: 32678 or a variant or homolog having substantial identity thereto.

A “EpCAM” or “EpCAM protein” as referred to herein includes any of the recombinant or naturally-occurring forms of Epithelial cell adhesion molecule (EpCAM), or variants or homologs thereof that maintain EpCAM activity (e.g. within at least 50%, 80%, 90%, 95%, 96%, 97%, 98%, 99% or 100% activity compared to EpCAM). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EpCAM polypeptide. In embodiments, the EpCAM protein is substantially identical to the protein identified by the NCBI reference number GI: 15928632 or a variant or homolog having substantial identity thereto. In embodiments, the EpCAM protein is substantially identical to the protein identified by the NCBI reference number GI: 160266056 or a variant or homolog having substantial identity thereto. In embodiments, the EpCAM protein is substantially identical to the protein identified by the NCBI reference number GI: 218505670 or a variant or homolog having substantial identity thereto.

A “CD19” or “CD19 protein” as referred to herein includes any of the recombinant or naturally-occurring forms of B-lymphocyte antigen CD19 (CD19) also known as Cluster of Differentiation 19, or variants or homologs thereof that maintain CD19 activity (e.g. within at least 50%, 80%, 90%, 95%, 96%, 97%, 98%, 99% or 100% activity compared to CD19). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 polypeptide. In embodiments, the CD19 protein is substantially identical to the protein identified by the NCBI reference number GI: 180034 or a variant or homolog having substantial identity thereto. In embodiments, the CD19 protein is substantially identical to the protein identified by the NCBI reference number GI: 901823 or a variant or homolog having substantial identity thereto. In embodiments, the CD19 protein is substantially identical to the protein identified by the NCBI reference number GI: 14495156 or a variant or homolog having substantial identity thereto.

A “GFP” or “GFP protein” as referred to herein includes any of the recombinant or naturally-occurring forms of Green Fluorescent Protein (GFP), or variants or homologs thereof that maintain GFP activity (e.g. within at least 50%, 80%, 90%, 95%, 96%, 97%, 98%, 99% or 100% activity compared to GFP). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GFP polypeptide. In embodiments, the GFP protein is substantially identical to the protein identified by the NCBI reference number GI: 262348071 or a variant or homolog having substantial identity thereto. In embodiments, the GFP protein is substantially identical to the protein identified by the NCBI reference number GI: 262348071 or a variant or homolog having substantial identity thereto. In embodiments, the GFP protein is substantially identical to the protein identified by the NCBI reference number GI: 1558075947 or a variant or homolog having substantial identity thereto.

A “luciferase” or “luciferase protein” as referred to herein includes any of the recombinant or naturally-occurring forms of firefly luciferase (luc), or variants or homologs thereof that maintain luciferase activity (e.g. within at least 50%, 80%, 90%, 95%, 96%, 97%, 98%, 99% or 100% activity compared to luciferase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring luciferase polypeptide. In embodiments, the luciferase protein is substantially identical to the protein identified by the NCBI reference number GI: 115342896 or a variant or homolog having substantial identity thereto. In embodiments, the luciferase protein is substantially identical to the protein identified by the NCBI reference number GI: 115342888 or a variant or homolog having substantial identity thereto. In embodiments, the luciferase protein is substantially identical to the protein identified by the NCBI reference number GI: 7981033 or a variant or homolog having substantial identity thereto.

The term “fragment,” as used herein, means a portion of a polypeptide or polynucleotide that is less than the entire polypeptide or polynucleotide. As used herein, a “functional fragment” of a protein, e.g., EGF, CEA, TAG-72, is a fragment of the polypeptide that is shorter than the full-length, immature, or mature polypeptide and has at least 25% (e.g., at least 30%, 40%, 50%, 60%, 70%, 80%, 90%, 95%, 98%, 99%, or even 100% or more) of the activity of full-length mature reference protein. Fragments of interest can be made by recombinant, synthetic, or proteolytic digestive methods.

A “ligand” refers to an agent, e.g., a polypeptide or other molecule, capable of binding to a receptor.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “autologous immune cell” as used herein refers to immune cells derived from the an individual that is typically both donor and recipient of the immune cells. An autologous immune cell is derived from the same individual and hence is genetically identical to the host with the exception of any ex-vivo changes made to the autologous immune cell. In embodiments, autologous immune cells are removed from a subject's body, cultured, activated, coated with dbBiTEs, and administered to the subject.

The term “genetically engineered immune cell” as used herein refers to altering the DNA in an immune cell. For example, a transfer of genes into the immune cell is used to produce a genetically engineered immune cell. For example, one base pair modification is used to produce a genetically engineered immune cell. For example, extracting DNA from another organism's genome and combining it with DNA of that individual is used to produce a genetically engineered immune cell. The DNA may be either isolated and copied or artificially synthesized. A construct is usually created and used to insert this DNA into the host cell. The construct may include a promoter and terminator region, which initiate and end transcription. The gene may also be modified for better expression or effectiveness. Modifications of the gene may be carried out using recombinant DNA techniques, such as restriction digests, ligations and molecular cloning. A number of techniques known in the art may be used to insert genetic material into the host genome of the immune cell. In embodiments, a genetically engineered immune cell may be a CAR T cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning. A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, an antibody and an antigen or a ligand and a receptor. In embodiments, contacting includes, for example, allowing an antibody (e.g., anti-CD6 antibody) to bind an antigen (e.g., CD6). In embodiments, contacting includes allowing a peptide (e.g., EGF) to bind to a receptor (e.g., EGFR).

The term “modulation”, “modulate”, or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target.

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In embodiments inhibition refers to reduction of a disease or symptoms of disease. Thus, in embodiments, inhibition includes, at least in part, partially or totally blocking stimulation, decreasing, preventing, or delaying activation, or inactivating, desensitizing, or down-regulating signal transduction or enzymatic activity or the amount of a protein. The amount of inhibition may be 10%, 20%, 30%, 40%, 50%, 60%, 70%, 80%, 90%, 100% or less in comparison to a control in the absence of the antagonist. In embodiments, the inhibition is 1.5-fold, 2-fold, 3-fold, 4-fold, 5-fold, 10-fold, or more than the expression or activity in the absence of the antagonist.

As defined herein, the term “activation”, “activate”, “activating” and the like in reference to a protein-activator (e.g. agonist) interaction means positively affecting (e.g. increasing) the activity or function of the relative to the activity or function of the protein in the absence of the activator. Thus, in embodiments, activation may include, at least in part, partially or totally increasing stimulation, increasing or enabling activation, or activating, sensitizing, or up-regulating signal transduction or enzymatic activity or the amount of a protein decreased in a disease. The amount of activation may be 10%, 20%, 30%, 40%, 50%, 60%, 70%, 80%, 90%, 100% or more in comparison to a control in the absence of the agonist. In embodiments, the activation is 1.5-fold, 2-fold, 3-fold, 4-fold, 5-fold, 10-fold, or more than the expression or activity in the absence of the agonist.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated activity (e.g. by using a method as described herein), results in reduction of the disease or one or more disease symptoms.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living member of the animal kingdom suffering from or that may suffer from the indicated disorder. In embodiments, the subject is a member of a species that includes individuals who naturally suffer from the disease. In embodiments, a subject is a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is an autoimmune disease (e.g. Type I Diabetes).

An “autoimmune disease” as used herein refers to a disease or disorder that arises from altered immune reactions by the immune system of a subject, e.g., against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, and allergic asthma.

As used herein, an “inflammatory disease” refers to a disease or disorder associated with abnormal or altered inflammation. Inflammation is a biological response initiated by the immune system as part of the healing process in response to a pathogen, damaged cells or tissues or irritants. Chronic inflammation can lead to a variety of diseases. Inflammatory diseases include, but are not limited to, atherosclerosis, allergies, asthma, rheumatoid arthritis, transplant rejection, celiac disease, chronic prostatitis, inflammatory bowel diseases, pelvic inflammatory diseases, and inflammatory myopathies.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., Type I Diabetes) means that the disease (e.g. Type I Diabetes) is caused by (in whole or in part), or a symptom of the disease is caused by (in whole or in part) the substance or substance activity or function.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of an autoimmune disease.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

### I. Methods of Treatment

Provided herein, inter alia, are methods of using bispecific antibody compounds which may bind immune cells and are, inter alia, useful for treating cancers. Anti-tumor antigen antibodies are connected to anti-immune antibodies through their hinge regions using, for example, click chemistry to form dual-specific, bivalent BiTES with high in vivo tumor targeting ability and tumor cytotoxicity. In an aspect is provided a method of treating cancer in a subject in need thereof, the method including administering to a subject a therapeutically effective amount of an autologous immune cell bound to a bispecific antibody compound as provided herein including embodiments thereof, thereby treating cancer in the subject. The bispecific antibody compound includes an anti-immune cell antibody covalently bound to an anti-cancer antibody through a linker. The linker is attached to a first amino acid within a first hinge region of the anti-immune cell antibody and a second amino acid within a second hinge region of the anti-cancer antibody.

In embodiments, the anti-immune cell antibody is non-covalently bound to an autologous immune cell. In embodiments, the autologous immune cell is an autologous T cell. In embodiments, the autologous immune cell is an autologous NK cell. In embodiments, the autologous immune cell is an autologous monocyte. In embodiments, the autologous immune cell is an autologous neutrophil. In embodiments, the autologous immune cell is an autologous macrophage. In embodiments, the autologous immune cell is a genetically engineered immune cell. In embodiments, autologous immune cell is an autologous T cell, autologous NK cell, autologous monocyte, autologous neutrophil, autologous macrophage, or a genetically engineered immune cell. In embodiments, the genetically engineered immune cell is a CAR T cell. In embodiments, the anti-immune cell antibody is non-covalently bound to a protein on the autologous immune cell.

In embodiments, the anti-cancer antibody is non-covalently bound to a cancer protein on a cancer cell. In embodiments, the cancer protein is carcinoembryonic antigen (CEA). In embodiments, the cancer protein is tumor-associated glycoprotein 72 (TAG-72). In embodiments, the cancer protein is epidermal growth factor receptor (EGFR) protein. In embodiments, the cancer protein is CD19. In embodiments, the cancer protein is CD20. In embodiments, the cancer protein is epithelial cell adhesion molecule (EpCAM). In embodiments, the cancer protein is human epidermal growth factor receptor 2 (Her2/neu). In embodiments, the cancer protein is carcinoembryonic antigen (CEA), tumor-associated glycoprotein 72 (TAG-72), epidermal growth factor receptor (EGFR) protein, CD19, CD20, epithelial cell adhesion molecule (EpCAM), or human epidermal growth factor receptor 2 (Her2/neu).

In embodiments the cancer is colorectal cancer. In embodiments the cancer is ovarian cancer. In embodiments the cancer is lung cancer. In embodiments the cancer is lymphoma. In embodiments the cancer is leukemia. In embodiments the cancer is breast cancer. In embodiments, the cancer is colorectal cancer, ovarian cancer, lung cancer, lymphoma, leukemia, or breast cancer.

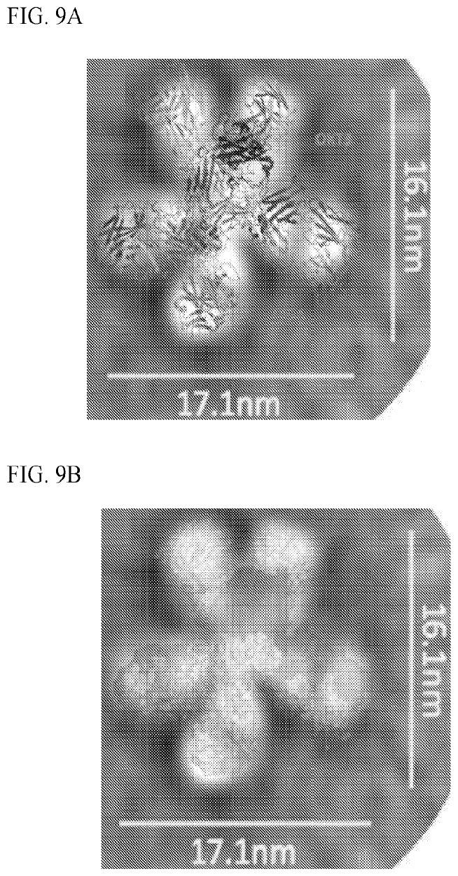

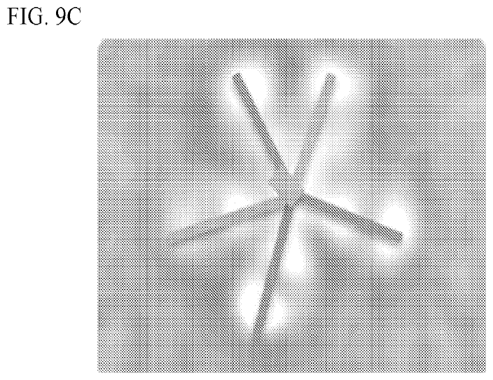

In embodiments, the first Fc domain of the anti-immune cell antibody is oriented in a direction different from the second Fc domain of the anti-cancer antibody. In embodiments, the first Fc domain of the anti-immune cell antibody is oriented in a direction approximately opposite of the second Fc domain of the anti-cancer antibody. In embodiments, the first Fc domain of the anti-immune cell antibody is oriented about 90°-180° from the second Fc domain of the anti-cancer antibody. In embodiments, the first Fc domain of the anti-immune cell antibody is oriented about 100°-180° from the second Fc domain of the anti-cancer antibody. In embodiments, the first Fc domain of the anti-immune cell antibody is oriented about 110°-180° from the second Fc domain of the anti-cancer antibody. In embodiments, the first Fc domain of the anti-immune cell antibody is oriented about 120°-180° from the second Fc domain of the anti-cancer antibody. In embodiments, the first Fc domain of the anti-immune cell antibody is oriented about 130°-180° from the second Fc domain of the anti-cancer antibody. In embodiments, the first Fc domain of the anti-immune cell antibody is oriented about 140°-180° from the second Fc domain of the anti-cancer antibody. In embodiments, the first Fc domain of the anti-immune cell antibody is oriented about 150°-180° from the second Fc domain of the anti-cancer antibody. In embodiments, the first Fc domain of the anti-immune cell antibody is oriented about 160-180° from the second Fc domain of the anti-cancer antibody. In embodiments, the first Fc domain of the anti-immune cell antibody is oriented about 170°-180° from the second Fc domain of the anti-cancer antibody. In embodiments, the first Fc domain of the anti-immune cell antibody is oriented about 90°-170° from the second Fc domain of the anti-cancer antibody. In embodiments, the first Fc domain of the anti-immune cell antibody is oriented about 90°-160° from the second Fc domain of the anti-cancer antibody. In embodiments, the first Fc domain of the anti-immune cell antibody is oriented about 90°-150° from the second Fc domain of the anti-cancer antibody. In embodiments, the first Fc domain of the anti-immune cell antibody is oriented about 90°-140° from the second Fc domain of the anti-cancer antibody. In embodiments, the first Fc domain of the anti-immune cell antibody is oriented about 90°-130° from the second Fc domain of the anti-cancer antibody. In embodiments, the first Fc domain of the anti-immune cell antibody is oriented about 90°-120° from the second Fc domain of the anti-cancer antibody. In embodiments, the first Fc domain of the anti-immune cell antibody is oriented about 90°-110° from the second Fc domain of the anti-cancer antibody. In embodiments, the first Fc domain of the anti-immune cell antibody is oriented about 90°-100° from the second Fc domain of the anti-cancer antibody. In embodiments, the first Fc domain of the anti-immune cell antibody is oriented about 90°, about 100°, about 110°, about 120°, about 130°, about 140°, about 150°, about 160°, about 170° or about 180° from the second Fc domain of the anti-cancer antibody. In embodiments, the first Fc domain of the anti-immune cell antibody is oriented 90°, 100°, 110°, 120°, 130°, 140°, 150°, 160°, 170° or 180° from the second Fc domain of the anti-cancer antibody. In embodiments, the bispecific antibody compound (dbBiTE) has a 6-lobe morphology. A “6-lobe morphology” as provided herein refers to a stereochemical structure which includes 6 lobes formed by the juxtaposition of each, two paratopes (antigen-binding site) and one Fc C-terminus, of two antibodies, wherein the antibodies are connected through any of the methods described herein (e.g., through click chemistry). An example of a 6-lobe morphology contemplated for the compositions and methods provided herein is depicted, for example, in FIGS. 9A-C. A “paratope” as described herein refers to the three-dimension structural domain of an antibody formed by the light chain variable domain and the heavy chain variable domain. The paratope or antigen-binding site is formed at the N-terminus of an antibody, an antibody variant or fragment thereof.

In embodiments, the anti-immune cell antibody has a molecular weight of about 75-200 kDa. In embodiments, the anti-immune cell antibody has a molecular weight of about 100-200 kDa. In embodiments, the anti-immune cell antibody has a molecular weight of about 125-200 kDa. In embodiments, the anti-immune cell antibody has a molecular weight of about 150-200 kDa. In embodiments, the anti-immune cell antibody has a molecular weight of about 175-200 kDa. In embodiments, the anti-immune cell antibody has a molecular weight of about 75-175 kDa. In embodiments, the anti-immune cell antibody has a molecular weight of about 75-150 kDa. In embodiments, the anti-immune cell antibody has a molecular weight of about 75-125 kDa. In embodiments, the anti-immune cell antibody has a molecular weight of about 75-100 kDa. In embodiments, the anti-immune cell antibody has a molecular weight of about 200 kDa, about 175 kDa, about 150 kDa, about 125 kDa, about 100 kDa, or about 75 kDa. In embodiments, the anti-immune cell antibody has a molecular weight of 200 kDa, 175 kDa, 150 kDa, 125 kDa, 100 kDa, or 75 kDa.

In embodiments, the anti-cancer cell antibody has a molecular weight of about 75-200 kDa. In embodiments, the anti-cancer cell antibody has a molecular weight of about 100-200 kDa. In embodiments, the anti-cancer cell antibody has a molecular weight of about 125-200 kDa. In embodiments, the anti-cancer cell antibody has a molecular weight of about 150-200 kDa. In embodiments, the anti-cancer cell antibody has a molecular weight of about 175-200 kDa. In embodiments, the anti-cancer cell antibody has a molecular weight of about 75-175 kDa. In embodiments, the anti-cancer cell antibody has a molecular weight of about 75-150 kDa. In embodiments, the anti-cancer cell antibody has a molecular weight of about 75-125 kDa. In embodiments, the anti-cancer cell antibody has a molecular weight of about 75-100 kDa. In embodiments, the anti-cancer cell antibody has a molecular weight of about 200 kDa, about 175 kDa, about 150 kDa, about 125 kDa, about 100 kDa, or about 75 kDa. In embodiments, the anti-cancer antibody has a molecular weight of 200 kDa, 175 kDa, 150 kDa, 125 kDa, 100 kDa, or 75 kDa.

In embodiments, the dbBiTE has a molecular weight of about 150-400 kDa. In embodiments, the dbBiTE has a molecular weight of about 175-400 kDa. In embodiments, the dbBiTE has a molecular weight of about 200-400 kDa. In embodiments, the dbBiTE has a molecular weight of about 225-400 kDa. In embodiments, the dbBiTE has a molecular weight of about 250-400 kDa. In embodiments, the dbBiTE has a molecular weight of about 275-400 kDa. In embodiments, the dbBiTE has a molecular weight of about 300-400 kDa. In embodiments, the dbBiTE has a molecular weight of about 325-400 kDa. In embodiments, the dbBiTE has a molecular weight of about 350-400 kDa. In embodiments, the dbBiTE has a molecular weight of about 375-400 kDa. In embodiments, the dbBiTE has a molecular weight of about 150-375 kDa. In embodiments, the dbBiTE has a molecular weight of about 150-350 kDa. In embodiments, the dbBiTE has a molecular weight of about 150-325 kDa. In embodiments, the dbBiTE has a molecular weight of about 150-300 kDa. In embodiments, the dbBiTE has a molecular weight of about 150-275 kDa. In embodiments, the dbBiTE has a molecular weight of about 150-250 kDa. In embodiments, the dbBiTE has a molecular weight of about 150-225 kDa. In embodiments, the dbBiTE has a molecular weight of about 150-200 kDa. In embodiments, the dbBiTE has a molecular weight of about 150-175 kDa. In embodiments, the dbBiTE has a molecular weight of about 400 kDa, about 375 kDa, about 350 kDa, about 325 kDa, about 300 kDa, about 375 kDa, about 350 kDa, about 325 kDa, about 300 kDa, about 275 kDa, about 250 kDa, about 225 kDa, about 200 kDa, about 175 kDa, or about 150 kDa. In embodiments, the dbBiTE has a molecular weight of 400 kDa, 375 kDa, 350 kDa, 325 kDa, 300 kDa, 375 kDa, 350 kDa, 325 kDa, 300 kDa, 275 kDa, 250 kDa, 225 kDa, 200 kDa, 175 kDa, or 150 kDa.

In embodiments, dbBiTEs include two intact antibodies each of which retains both Fab′ domains. In embodiments, dbBiTEs include two intact antibodies each of which retains bivalent binding ability to antigens.

In embodiments, the anti-cancer antibody is covalently attached to a detection moiety. In embodiments, the anti-cancer antibody is a targeting moiety. In embodiments, the detection moiety is Cu 64-DOTA. In embodiments, the anti-cancer antibody attached to a detection moiety is used for immunoPET studies. In embodiments, the anti-cancer antibody is covalently attached to a detection moiety is used for quantitative immunoPET studies. In embodiments, the anti-cancer antibody attached to Cu 64-DOTA is used for immunoPET studies. In embodiments, the anti-cancer antibody covalently attached to Cu 64-DOTA is used for quantitative immunoPET studies.

In embodiments, the linker has the formula -L¹-L²-L³-L⁴-L⁵-wherein, L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L¹ is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L¹ is independently a bond, —O—, —S—, —NH—, —C(O)—, —C(O)O—, —C(O)NH—, —OC(O)—, —NHC(O)—, —NH—C(O)—NH—, —OC(O)NH—, —NHC(O)O—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group. In embodiments, where the substituted L¹ is substituted with a plurality of substituent groups, each substituent group group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L² is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L² is independently a bond, —O—, —S—, —NH—, —C(O)—, —C(O)O—, —C(O)NH—, —OC(O)—, —NHC(O)—, —NH—C(O)—NH—, —OC(O)NH—, —NHC(O)O—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group. In embodiments, where the substituted L² is substituted with a plurality of substituent groups, each substituent group group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L³ is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L³ is independently a bond, —O—, —S—, —NH—, —C(O)—, —C(O)O—, —C(O)NH—, —OC(O)—, —NHC(O)—, —NH—C(O)—NH—, —OC(O)NH—, —NHC(O)O—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group. In embodiments, where the substituted L³ is substituted with a plurality of substituent groups, each substituent group group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L⁴ is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene

In embodiments, L⁴ is independently a bond, —O—, —S—, —NH—, —C(O)—, —C(O)O—, —C(O)NH—, —OC(O)—, —NHC(O)—, —NH—C(O)—NH—, —OC(O)NH—, —NHC(O)O—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group. In embodiments, where the substituted L⁴ is substituted with a plurality of substituent groups, each substituent group group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, L⁵ is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L⁵ is independently a bond, —O—, —S—, —NH—, —C(O)—, —C(O)O—, —C(O)NH—, —OC(O)—, —NHC(O)—, —NH—C(O)—NH—, —OC(O)NH—, —NHC(O)O—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, a substituted L⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group. In embodiments, where the substituted L⁵ is substituted with a plurality of substituent groups, each substituent group group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, L¹ and L⁵ are —CH₂—S—. In embodiments, L¹ includes —CH₂—S—. In embodiments, L¹ is —CH₂—S—. In embodiments, L⁵ includes —CH₂—S—. In embodiments, L⁵ is —CH₂—S—. In embodiments, L³ includes azocinylene. In embodiments, L³ is azocinylene. In embodiments, L³ is 3a,8,9,13b-tetrahydro-1H-dibenzo[b,f][1,2,3]triazolo[4,5-d]azocine.

### II. Processes of Making Cell Compositions

In another aspect is provided a process for making an autologous immune cell bound to a bispecific antibody compound (dbBiTE), as provided herein including embodiments thereof. The process includes combining an autologous immune cell and an anti-immune cell antibody in a reaction vessel thereby forming an autologous immune cell-antibody complex. The autologous immune cell-antibody complex includes the autologous immune cell bound to the anti-immune cell antibody, wherein the anti-immune cell antibody includes a first hinge region amino acid and the first hinge region amino acid includes a first reactive chemical group side chain. The process further includes combining the autologous immune cell-antibody complex with a second antibody in a reaction vessel thereby forming the autologous immune cell bound to the bispecific antibody compound. The second antibody includes a second hinge region amino acid and the second hinge region amino acid includes a second reactive chemical group side chain that is reactive with the first reactive chemical group side chain. The bispecific antibody compound includes the anti-immune cell antibody covalently bound to the second antibody through a linker, wherein the linker is attached to the first hinge region amino acid and the second hinge region amino acid.

In embodiments, the first reactive chemical group side chain has the formula -L¹-L²-R¹. L¹ and L² are as defined above, including embodiments thereof. In embodiments, R¹ is a first reactive functional group. In embodiments R¹ is a bioconjugate reactive group. In embodiments R¹ is a click chemistry reactive functional group. In embodiments, R¹ includes -dibenzocyclooctyl (DBCO). In embodiments, R¹ is -DBCO. In embodiments, R¹ includes —N₃. In embodiments, R¹ is —N₃.

In embodiments, the second reactive chemical group side chain has the formula -L⁵-L⁴-R². L⁴ and L⁵ are as defined above, including embodiments thereof. In embodiments, R² is a second reactive functional group complementary to the first reactive functional group. In embodiments R² is a bioconjugate reactive group. In embodiments R² is a click chemistry reactive functional group. In embodiments, R² includes -DBCO. In embodiments, R² is -DBCO. In embodiments, R² includes —N₃. In embodiments, R² is —N₃.

In embodiments, the linker has the formula -L¹-L²-L³-L⁴-L⁵-. L¹, L², L³, L⁴, and L⁵ are as defined above, including embodiments thereof.

In embodiments, the anti-immune cell antibody is non-covalently bound to the autologous immune cell. In embodiments, the autologous immune cell is an autologous T cell. In embodiments, the autologous immune cell is an autologous NK cell. In embodiments, the autologous immune cell is an autologous monocyte. In embodiments, the autologous immune cell is an autologous neutrophil. In embodiments, the autologous immune cell is an autologous macrophage. In embodiments, the autologous immune cell is a genetically engineered immune cell. In embodiments, autologous immune cell is an autologous T cell, autologous NK cell, autologous monocyte, autologous neutrophil, autologous macrophage, or a genetically engineered immune cell. In embodiments, the genetically engineered immune cell is a CAR T cell. In embodiments, the anti-immune cell antibody is non-covalently bound to a protein on the autologous immune cell.

In embodiments, the second antibody is an anti-cancer antibody. In embodiments the cancer is colorectal cancer. In embodiments the cancer is ovarian cancer. In embodiments the cancer is lung cancer. In embodiments the cancer is lymphoma. In embodiments the cancer is leukemia. In embodiments the cancer is breast cancer. In embodiments, the cancer is colorectal cancer, ovarian cancer, lung cancer, lymphoma, leukemia, or breast cancer. In embodiments, the second antibody is covalently attached to a detection moiety.

In embodiments, the first Fc domain of the anti-immune cell antibody is oriented in a direction opposite of the second Fc domain of the second antibody. In embodiments, the first Fc domain of the anti-immune cell antibody is oriented about 90°-180° from the second Fc domain of the second antibody. In embodiments, the first Fc domain of the anti-immune cell antibody is oriented about 90°, about 100°, about 110°, about 120°, about 130°, about 140°, about 150°, about 160°, about 170° or about 180° from the second Fc domain of the second antibody. In embodiments, the first Fc domain of the anti-immune cell antibody is oriented 90°, 100°, 110°, 120°, 130°, 140°, 150°, 160°, 70° or 180° from the second Fc domain of the second antibody. In embodiments the bispecific antibody compound has a 6-lobe morphology.

In embodiments, the anti-immune cell antibody has a molecular weight of about 75-200 kDa. In embodiments, the anti-immune cell antibody has a molecular weight of about 200 kDa, about 175 kDa, about 150 kDa, about 125 kDa, about 100 kDa, or about 75 kDa. In embodiments, the anti-immune cell antibody has a molecular weight of 200 kDa, 175 kDa, 150 kDa, 125 kDa, 100 kDa, or 75 kDa.

In embodiments, the second antibody has a molecular weight of about 75-200 kDa. In embodiments, the second antibody has a molecular weight of about 200 kDa, about 175 kDa, about 150 kDa, about 125 kDa, about 100 kDa, or about 75 kDa. In embodiments, the second antibody has a molecular weight of 200 kDa, 175 kDa, 150 kDa, 125 kDa, 100 kDa, or 75 kDa.

In embodiments, the dbBiTE has a molecular weight of about 150-400 kDa. In embodiments, the dbBiTE has a molecular weight of about 400 kDa, about 375 kDa, about 350 kDa, about 325 kDa, about 300 kDa, about 375 kDa, about 350 kDa, about 325 kDa, about 300 kDa, about 275 kDa, about 250 kDa, about 225 kDa, about 200 kDa, about 175 kDa, or about 150 kDa. In embodiments, the dbBiTE has a molecular weight of 400 kDa, 375 kDa, 350 kDa, 325 kDa, 300 kDa, 375 kDa, 350 kDa, 325 kDa, 300 kDa, 275 kDa, 250 kDa, 225 kDa, 200 kDa, 175 kDa, or 150 kDa.

### III. Cell Compositions

In another aspect is provided an autologous immune cell bound to an anti-immune cell antibody. The anti-immune cell antibody includes a hinge region amino acid. The hinge region amino acid includes a reactive chemical group side chain. In embodiments, the the reactive chemical group side chain has the formula -L¹-L²-R¹. L¹ and L² are as defined above, including embodiments thereof. In embodiments, R¹ is a first reactive functional group. In embodiments R¹ is a bioconjugate reactive group. In embodiments R¹ is a click chemistry reactive functional group. In embodiments, R¹ includes -dibenzocyclooctyl (DBCO). In embodiments, R¹ is DBCO. In embodiments, R¹ includes —N₃. In embodiments, R¹ is —N₃.

In another aspect is provided an autologous immune cell bound to a bispecific antibody compound as provided herein, including embodiments thereof. The bispecific antibody compound includes an anti-immune cell antibody covalently bound to an anti-cancer antibody through a linker The linker is attached to a first amino acid within a first hinge region of the anti-immune cell antibody and a second amino acid within a second hinge region of the anti-cancer antibody.

In embodiments, the linker the linker has the formula -L¹-L²-L³-L⁴-L⁵-. L¹, L², L³, L⁴, and L⁵ are as defined above, including embodiments thereof. In embodiments, L¹ and L⁵ are —CH₂—S—. In an embodiment L¹ includes —CH₂—S—. In an embodiments L¹ is —CH₂—S—. In an embodiment L⁵ includes —CH₂—S—. In an embodiments L⁵ is —CH₂—S—. In embodiments, L³ is azocinylene. In embodiments, L³ is 3a,8,9,13b-tetrahydro-1H-dibenzo[b,f][1,2,3]triazolo[4,5-d]azocine.

n embodiments, the anti-immune cell antibody is non-covalently bound to the autologous immune cell. In embodiments, the autologous immune cell is an autologous T cell. In embodiments, the autologous immune cell is an autologous NK cell. In embodiments, the autologous immune cell is an autologous monocyte. In embodiments, the autologous immune cell is an autologous neutrophil. In embodiments, the autologous immune cell is an autologous macrophage. In embodiments, the autologous immune cell is a genetically engineered immune cell. In embodiments, autologous immune cell is an autologous T cell, autologous NK cell, autologous monocyte, autologous neutrophil, autologous macrophage, or a genetically engineered immune cell. In embodiments, the genetically engineered immune cell is a CAR T cell. In embodiments, the anti-immune cell antibody is non-covalently bound to a protein on the autologous immune cell.

In embodiments, the anti-cancer antibody is non-covalently bound to a cancer protein on a cancer cell. In embodiments, the cancer protein is carcinoembryonic antigen (CEA). In embodiments, the cancer protein is tumor-associated glycoprotein 72 (TAG-72). In embodiments, the cancer protein is epidermal growth factor receptor (EGFR) protein. In embodiments, the cancer protein is CD19. In embodiments, the cancer protein is CD20. In embodiments, the cancer protein is epithelial cell adhesion molecule (EpCAM). In embodiments, the cancer protein is human epidermal growth factor receptor 2 (Her2/neu). In embodiments, the cancer protein is carcinoembryonic antigen (CEA), tumor-associated glycoprotein 72 (TAG-72), epidermal growth factor receptor (EGFR) protein, CD19, CD20, epithelial cell adhesion molecule (EpCAM), or human epidermal growth factor receptor 2 (Her2/neu).

In embodiments the cancer is colorectal cancer. In embodiments the cancer is ovarian cancer. In embodiments the cancer is lung cancer. In embodiments the cancer is lymphoma. In embodiments the cancer is leukemia. In embodiments the cancer is breast cancer. In embodiments, the cancer is colorectal cancer, ovarian cancer, lung cancer, lymphoma, leukemia, or breast cancer.

In embodiments, the anti-cancer antibody is covalently attached to a detection moiety.

In embodiments, the first Fc domain of the anti-immune cell antibody is oriented in a direction opposite of the second Fc domain of the anti-cancer antibody. In embodiments, the first Fc domain of the anti-immune cell antibody is oriented about 90-180° from the second Fc domain of the anti-cancer antibody. In embodiments, the first Fc domain of the anti-immune cell antibody is oriented about 90°, about 100°, about 110°, about 120°, about 130°, about 140°, about 150°, about 160°, about 170° or about 180° from the second Fc domain of the anti-cancer antibody. In embodiments, the first Fc domain of the anti-immune cell antibody is oriented 90°, 100°, 110°, 120°, 130°, 140°, 150°, 160°, 170° or 180° from the second Fc domain of the anti-cancer antibody. In embodiments the bispecific antibody compound has a 6-lobe morphology.

In embodiments, the anti-immune cell antibody has a molecular weight of about 75-200 kDa. In embodiments, the anti-immune cell antibody has a molecular weight of about 200 kDa, about 175 kDa, about 150 kDa, about 125 kDa, about 100 kDa, or about 75 kDa. In embodiments, the anti-immune cell antibody has a molecular weight of 200 kDa, 175 kDa, 150 kDa, 125 kDa, 100 kDa, or 75 kDa.

In embodiments, the anti-cancer cell antibody has a molecular weight of about 75-200 kDa. In embodiments, the anti-cancer cell antibody has a molecular weight of about 200 kDa, about 175 kDa, about 150 kDa, about 125 kDa, about 100 kDa, or about 75 kDa. In embodiments, the anti-cancer antibody has a molecular weight of 200 kDa, 175 kDa, 150 kDa, 125 kDa, 100 kDa, or 75 kDa.

In embodiments, the dbBiTE has a molecular weight of about 150-400 kDa. In embodiments, the dbBiTE has a molecular weight of about 400 kDa, about 375 kDa, about 350 kDa, about 325 kDa, about 300 kDa, about 375 kDa, about 350 kDa, about 325 kDa, about 300 kDa, about 275 kDa, about 250 kDa, about 225 kDa, about 200 kDa, about 175 kDa, or about 150 kDa. In embodiments, the dbBiTE has a molecular weight of 400 kDa, 375 kDa, 350 kDa, 325 kDa, 300 kDa, 375 kDa, 350 kDa, 325 kDa, 300 kDa, 275 kDa, 250 kDa, 225 kDa, 200 kDa, 175 kDa, or 150 kDa.

### IV. Pharmaceutical Compositions

The bispecific antibody compound (dbBiTE) bound to an autologous immune cell provided herein, including embodiments thereof, may form part of a pharmaceutical composition. Thus, in embodiments, the composition is a pharmaceutical composition. In embodiments, the pharmaceutical composition includes a pharmaceutically acceptable excipient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Generation of Dual Specific Bivalent Bites for Cellular Immunotherapy

Bispecific T-cell engaging antibodies (BiTES), comprising dual anti-CD3 and anti-tumor antigen scFv fragments, are important therapeutic agents for the treatment of cancer. The dual scFv construct for BiTES requires proper protein folding while their small molecular size leads to rapid kidney clearance. Here applicants show that an intact (about 150 kDa) anti-tumor antigen antibody to CEA was joined in high yield (ca. 30%) to intact (about 150 kDa) anti-murine and anti-human CD3 antibodies using hinge region specific click chemistry to form dual-specific, bivalent BiTES (dbBiTES, with a molecular weight of about 300 kDa). The interlocked hinge regions are compatible with a structural model that fits the electron micrographs of the 300 kDa particles. Compared to intact anti-CEA antibody, dbBiTES maintain high in vivo tumor targeting as demonstrated by PET imaging, and redirect dbBiTE coated T-cells (1 microgram/10 million cells) to kill CEA+target cells both in vitro, and in vivo in CEA transgenic mice.

### Introduction

Directing T-cells to tumors with bispecific antibodies was first explored when recombinant DNA technologies made the genetic engineering of bispecific antibodies possible [1]. Today the most widely used BiTE (Bispecific T-cell Engaging antibodies) constructs join two single chain Fv fragments together with a linker to form a 50 kDa protein. Examples of tumor target antigens include CD19, EpCAM, Her2/neu, EGFR, CEA, and more [1]. The BiTE anti-CD19/anti-CD3 (Blinatumomab) has had remarkable success in the treatment of several B-cell malignancies, suggesting that directing T-cell (and other effector cells) to tumors by a bispecific antibody approach can be effective even against solid tumors [2]. Limitations of the therapy include the small molecular size that requires constant infusion of rather large amounts of BiTEs, the poly-activation of endogenous TCRs may lead to off target toxicity, and the lack of an engagement of a co-stimulus such as CD28 on the T-cell may limit effective tumor killing. An alternate approach pioneered by Lum and associates [3] utilized chemical conjugation with protein surface cross-linking agents on intact antibodies (eg., anti-CD3 and anti-CA125) that were coated onto IL-2 activated autologous T-cells. In spite of the high heterogeneity of the resulting bispecific antibodies, advantages of the approach were that only microgram amounts of antibody were required for coating the T-cells and the therapy was considered a cell-based therapy by the FDA since it involved infusion of T-cells and not antibody infusion. However, to bring the approach into widespread use, it is important to develop a high yield conjugation method that does not lead to a complex mixture of bispecific antibodies.

To overcome this challenge, Applicants show here an efficient approach to conjugate two antibodies alkylated at their sulfhydryl reduced hinge regions with two complimentary Click reagents, bromoacetamido-dibenzocyclooctyne (DBCO) and bromoacetamido-PEG₅-azide. To demonstrate feasibility, applicants selected the humanized anti-CEA antibody T84.66-M5A (M5A) and anti-CD3 antibody OKT3, both extensively used in the clinic [4, 5]. Given their dual specifities and retention of bivalent binding, applicants call them dbBiTEs. The 300 kDa product when purified away from contaminating 150 kDa species, shows distinctive six-lobed antibody particles on electron microscopy that fit atomic scale models, labels both CEA and CD3 positive cells, and when coated on activated T-cells, kills CEA positive cells in cytotoxicity assays. In vivo PET imaging shows excellent targeting to tumor targets, and in the preliminary therapy studies, killing of CEA positive tumors.

Results

Generation of dbBiTE

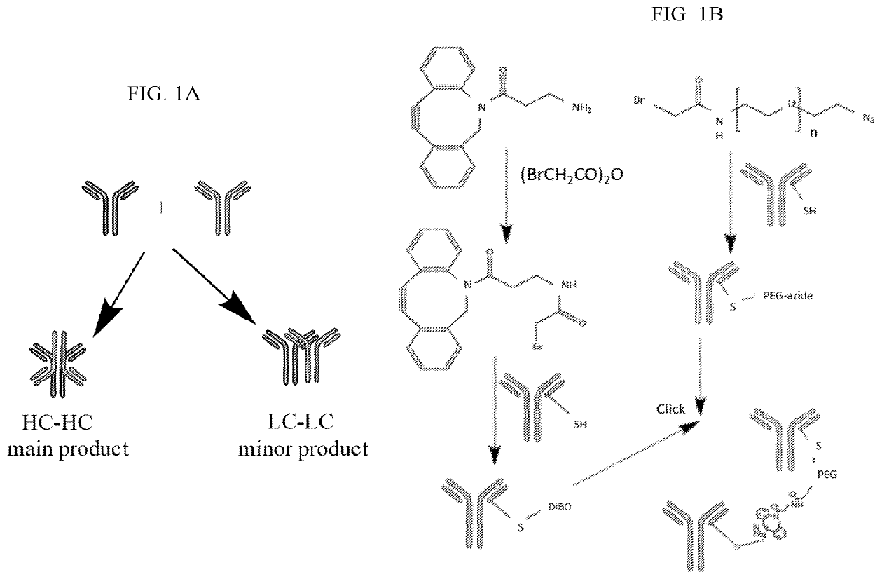

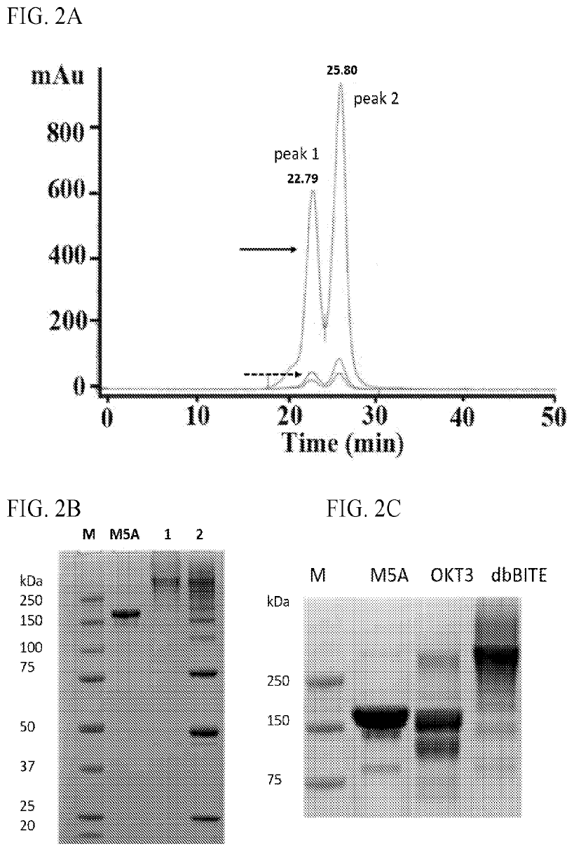

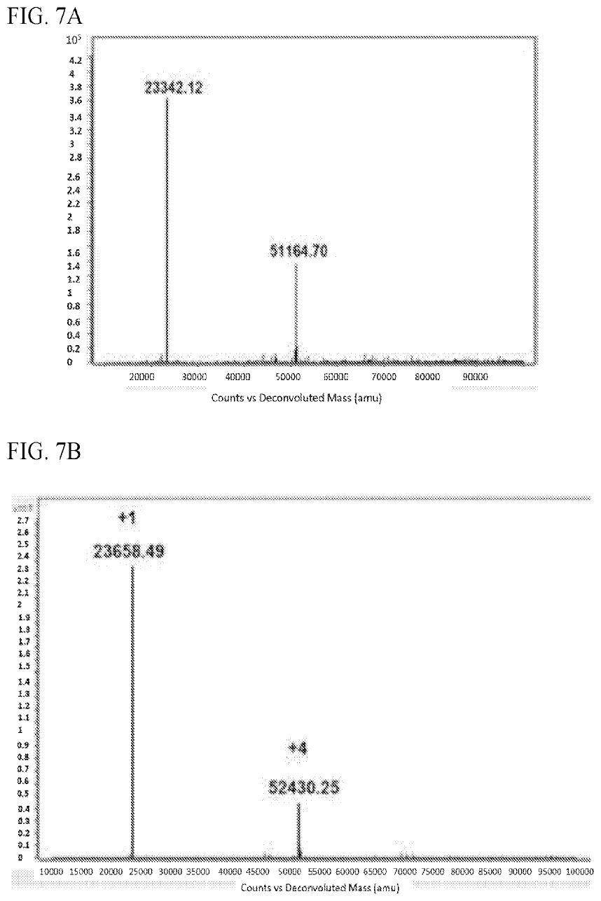

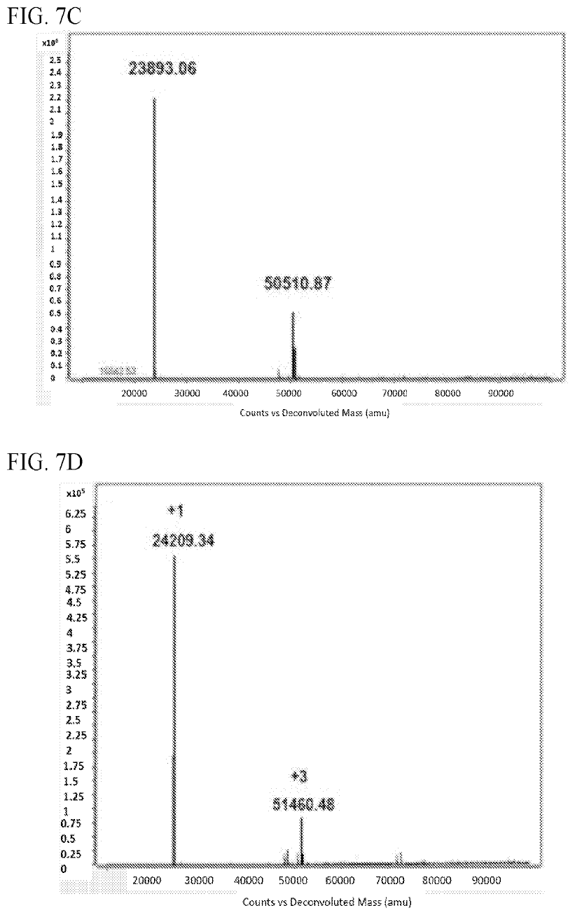

Applicants generated bispecific antibody compounds by cross-linking two intact antibodies at their hinge regions using Click chemistry (FIGS. 1A-B). A previous approach developed by Lum et al. [3] included randomly cross-linked two intact antibodies (anti-CD3 and anti-CA125) using Traut's reagent and sulfosuccinimidyl 4-(Nmaleimidomethyl) cyclohexane-1-carboxylate. The use of Click chemistry allows the generation of a bispecific antibody product with improved yields and molecular characteristics, a much-desired feature in bringing new products to the clinic. To distinguish the product from conventional BiTEs built from monovalent single chain Fv (scFv) fragments, applicants term the hinge region cross-linked two intact antibodies dbBiTE, for dual specific bivalent BiTE. Briefly, OKT3, a murine anti-human CD3 antibody widely used in the clinic [4] was reduced at its hinge region cysteines under non-denaturing conditions, alkylated at its reduced hinge region cysteines with a bromoacetamido-PEG₅-azido derivative and conjugated to a humanized anti-CEA M5A antibody [5] that was similarly reduced and alkylated with a bromoacetamido-DIBO derivative. Each of the derivatized IgGs were analyzed by electrospray ionization mass spectrometry to confirm their degree of derivatization (FIGS. 7A-D). In both cases the heavy chains contained at least 2 Click derivatives. As shown in FIG. 2A, the clicked dbBiTE was isolated in a yield of 30% by size exclusion chromatography (SEC, peak 1, 300 kDa). A second peak of MW 150 kDa was obtained in a yield of 60%. The peaks were analyzed by nondenaturing SDS gel electrophoresis to further verify their molecular sizes compared to the two intact antibodies (FIGS. 2B-C). The 150 kDa peak was shown to be mainly a monovalent bispecific antibody that was poorly active in functional studies (redirected T-cell cytotoxicity) and was broken down into lower molecular weight fragments by SDS gel electrophoresis. Therefore, applicants focused on the novel dbBiTE (300 kDa) since it retains the inherent avidity (bivalent binding) of both parent antibodies. Peak 1 was re-purified by SEC to remove contaminating peak 1. Re-chromatography of peak 1 over the course of several weeks showed no evidence of instability consistent with their covalent linkages.

Particle Size by Electron Microscopy.

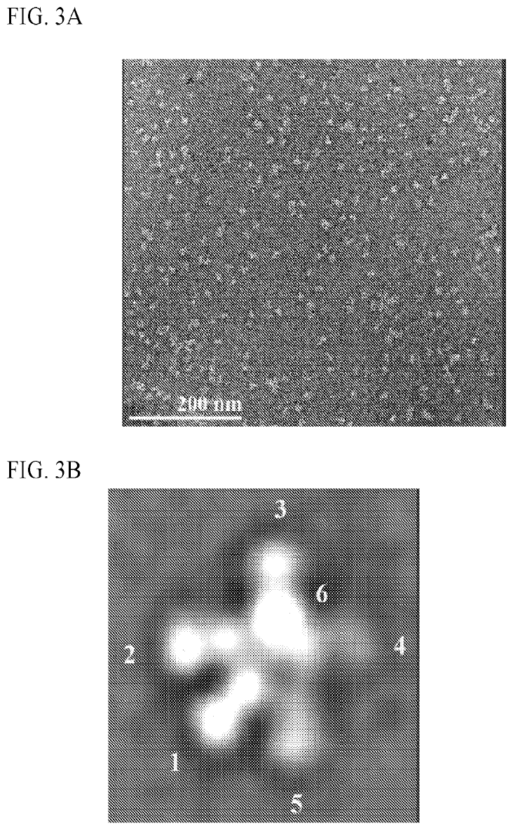

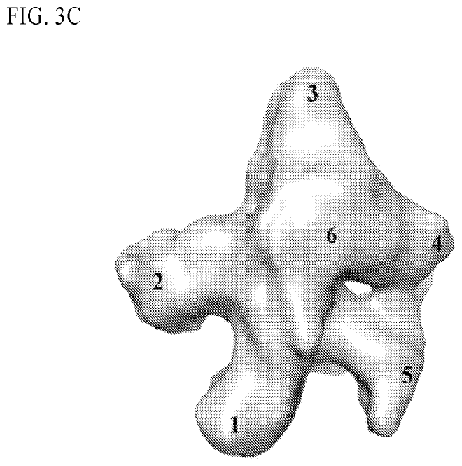

Purified peak 1 was analyzed by transmission electron microscopy (TEM) to determine the particle size and morphology (FIG. 3A). Most particles exhibited a 5-lobed morphology suggesting that the predicted sixth lobe (based on 3 lobes per Ab times 2) was hidden under or above the plane of the 2D image. This morphology is more clearly visualized on the 2D averaged analysis (FIG. 3B). Using tilt axis imaging, it was possible to show a representative 3D image (FIG. 3C). A closer examination of the particles shown in (FIG. 3A) reveals multiple orientations of 6-lobed particles, always with the sixth lobe appearing as additional density in the center of the particle. These images are compatible with the random landing of 6-lobed 3D particles on a 2D grid in which one lobe is lying above or below the main body of the particle. In addition to the majority of 6-lobed particles found, a small percentage (<10%) of particles with a side-to side orientation were observed. Applicants speculate that these are due to Click chemistry occurring between adjacent light chains (1 cysteine per light chain) at their hinge regions, rather than the more frequent heavy chain hinge regions (3 cysteines per heavy chain in human IgG1).

Molecular Simulations of dbBiTE Structure.

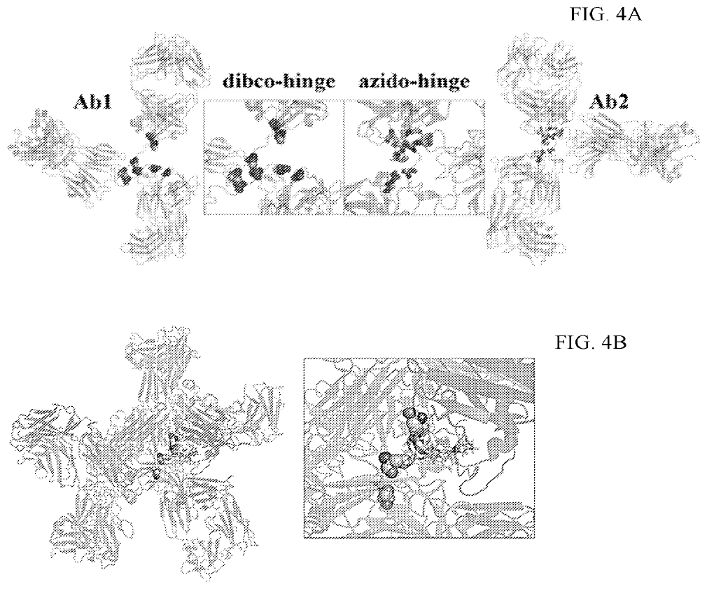

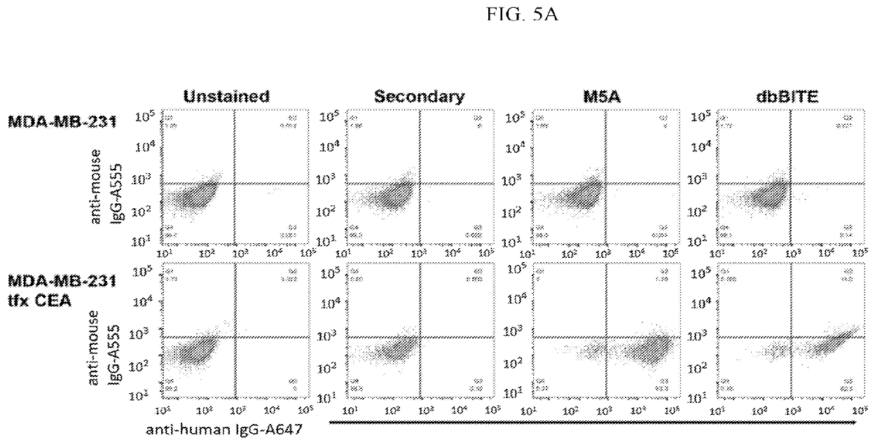

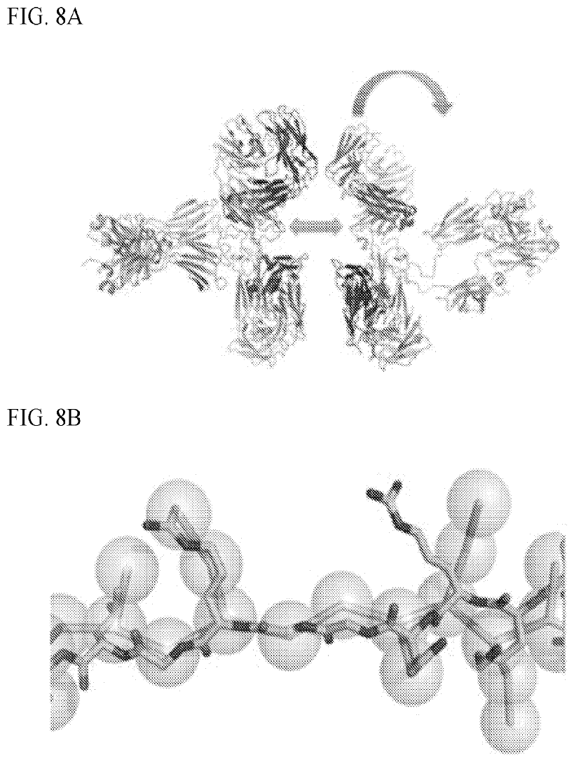

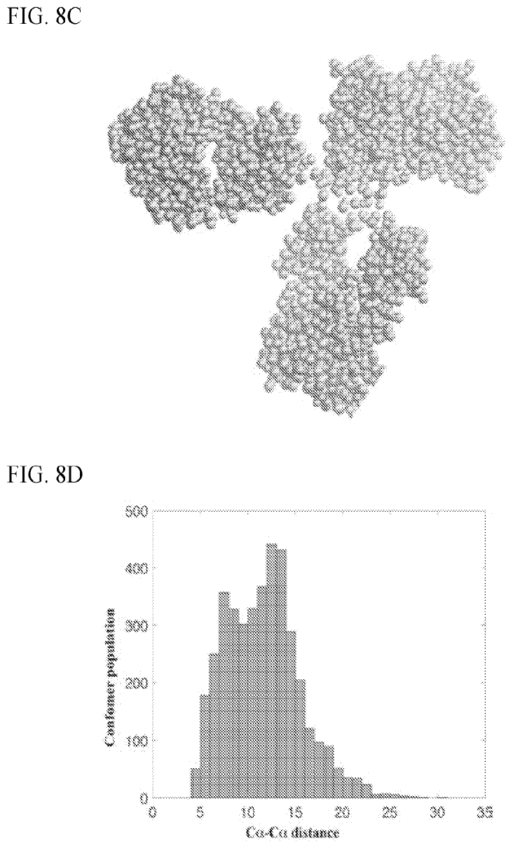

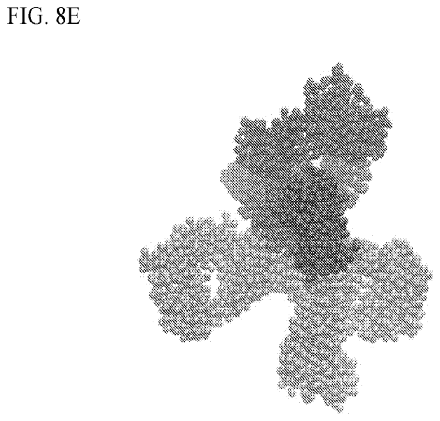

Applicants performed multi-scale molecular dynamics simulations to generate an atomic level structural model examining how the two IgGs fit into a dbBiTE joined by Click chemistry at their hinge region cysteines. Applicants generated a coarse grain homology model of the two IgGs (FIGS. 8A-E) that used the coarse grain simulation method Martini to optimize the packing of these two moieties. The optimized structural model shows the two IgGs joined by at least two pairs of Clicked hinge region cysteines (FIGS. 4A-B). The final docked dbBiTES were then oriented to fit within the observed 6-lobed particles found on EM (FIGS. 9A-C). The superposition of the dbBiTE structural model thus generated onto the particles imaged by EM strongly supports the idea that two IgGs can indeed be joined together at their hinge regions in spite of the size of their 3 globular domains.

In Vitro Binding and Cytotoxicity of dbBiTEs to CEA Positive Targets.

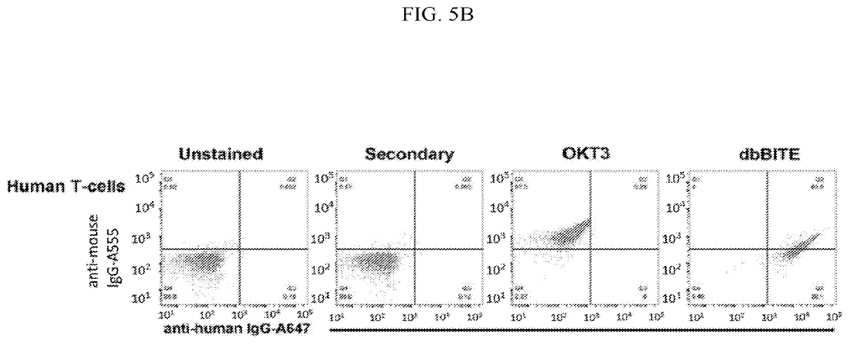

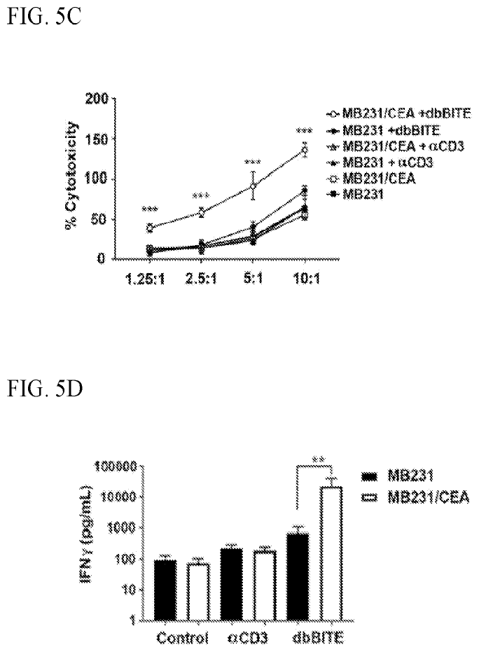

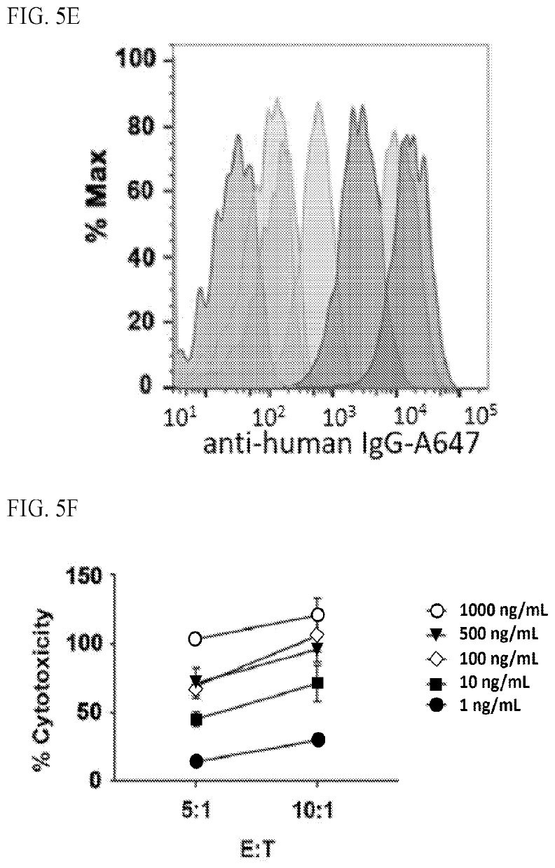

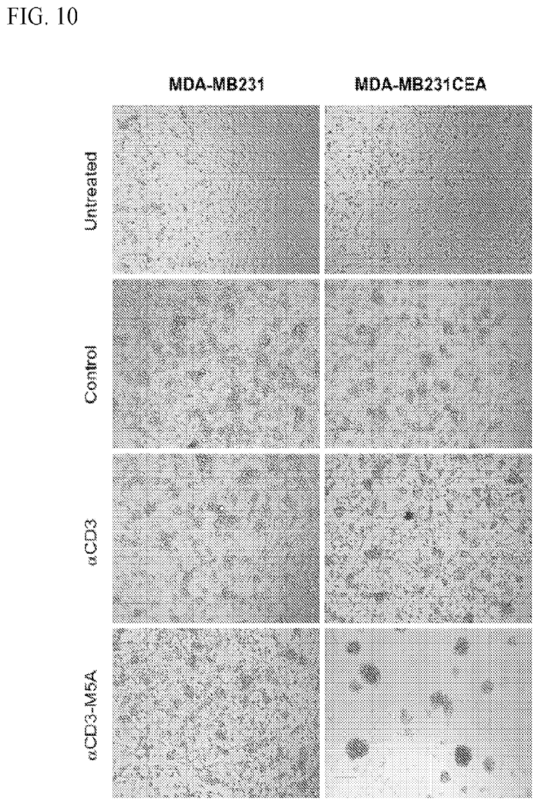

Since it was important to demonstrate that both antibody specificities were retained in the dbBiTE, in vitro binding studies were performed comparing the starting antibodies to the dbBiTE on CEA and CD3 positive targets. The results in FIGS. 5A-B demonstrate that dbBiTES are able to bind both CEA and CD3 positive target cells. In vitro cytotoxicity was demonstrated by coating activated human T-cells with dbBiTES (1 μg per 1×10⁶ cells per mL) and incubation with CEA positive targets at the indicated E:T ratios (FIG. 5C). Effective killing was observed as low as an E:T of 1.25:1 with maximal killing at an E:T of 10:1. Analysis of the supernatants revealed a significant release of IFNγ compared to controls (FIG. 5D) demonstrating that the dbBiTE coated activated T-cells were able to produce a functional cytokine in response to target engagement. When the coating capacity of activated T-cells with dbBiTEs was tested by flow analysis, it was found that as little as 1 ng/mL of dbBiTE incubated with 10M T-cells per mL was detectable (FIG. 5E). Although the cytotoxicity of activated T-cells against CEA positive targets was detectable at this concentration, higher coating concentrations were more effective (FIG. 5F). Microscopic images of the killing of CEA positive targets by dbBiTE coated activated T-cells are shown in FIG. 10. These results indicate activated T-cells coated with anti-CD3 alone caused some clumping and killing of targets, but dbBiTE coated T-cells completely killed all of the CEA+target cells compared to the CEA− target cells.

In Vivo Targeting of dbBiTEs to a CEA Positive Tumor in CEA Tg Mice

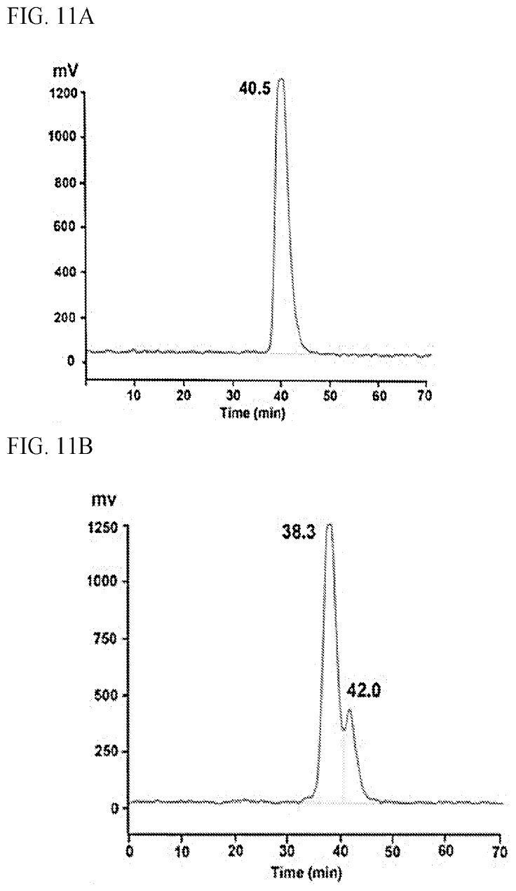

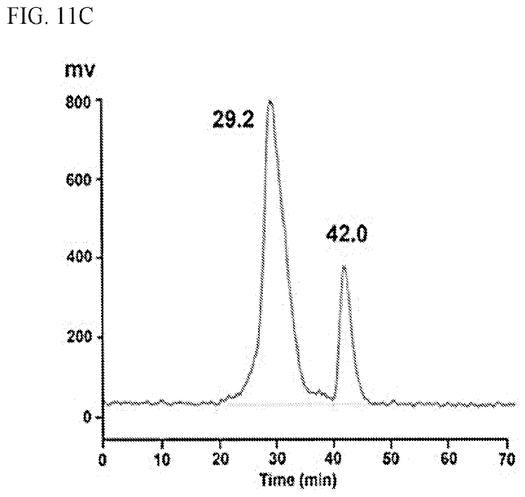

It was also important to demonstrate in vivo targeting of dbBiTEs to CEA positive tumors, since therapy studies were planned with dbBiTE coated T-cells. This study involved DOTA conjugated dbBiTEs labeled with ⁶⁴Cu for positron emission tomography (PET) of CEA positive human colon carcinoma LS174T xenografts in NOD/SCID mice. When radiolabeled dbBiTE (300 kDa) was compared to intact M5A (150 kDa) on SEC, dbBiTE was shown to be about 88% single species with about 12% molar contamination of a 150 kDa species (FIGS. 11A-C). The 150 kDa contaminant is likely a mixture of intact M5A and OKT3 or their Click conjugated half molecules. Since only the 300 kDa species shifted to a higher molecular complex on the addition of unlabeled CEA, applicants conclude that only the 300 kDa dbBiTE is immunoreactive.

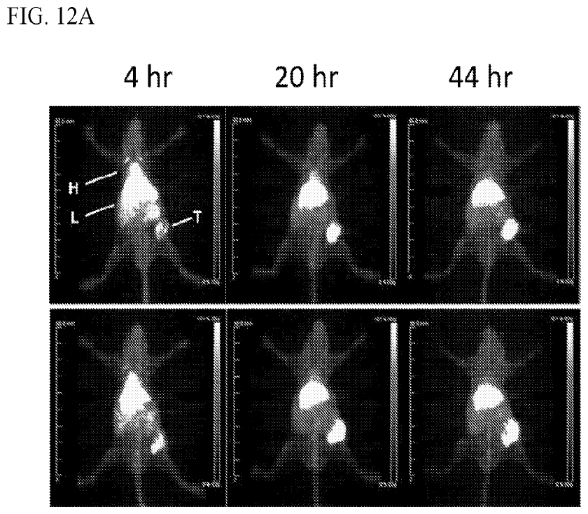

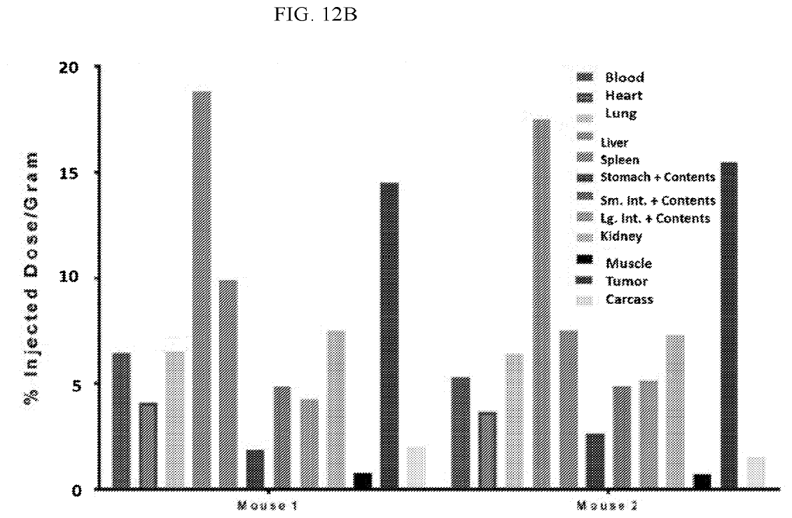

When ⁶⁴Cu-DOTA labeled dbBiTE was injected into CEA positive tumor bearing mice, PET imaging revealed rapid uptake into both tumor and liver with evidence of blood pool in the heart at the earliest time point (FIGS. 12A-B). At the terminal time of 44 hr, 15% ID/g was found in tumor, 5% injected dose per gram (ID/g) in blood and 18% ID/g in liver. Compared previous PET images of ⁶⁴Cu-DOTA labeled M5A that showed about 45% ID/g in tumor, 10% ID/g in blood and 12% ID/g in liver at 44 hrs [6], the faster clearance of dbBiTE from blood into liver may be due to liver recognition of the multiple Fc moieties on the dbBiTE. Nonetheless, the PET imaging demonstrates excellent targeting of the dbBiTE to tumor, encouraging applicants to test the efficacy of the dbBiTE in vivo.

In Vivo Killing of CEA Positive Tumors.

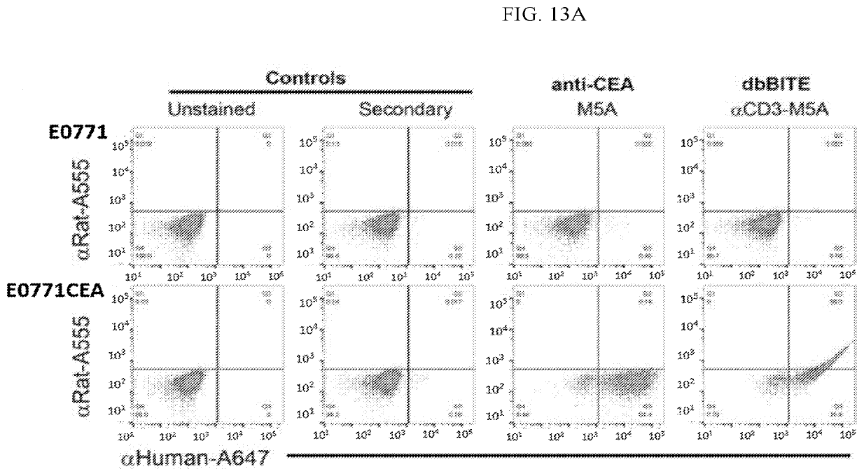

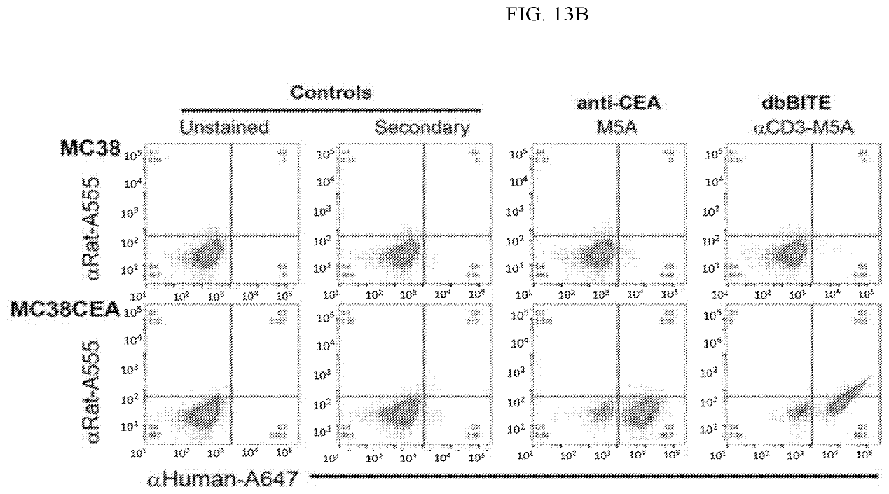

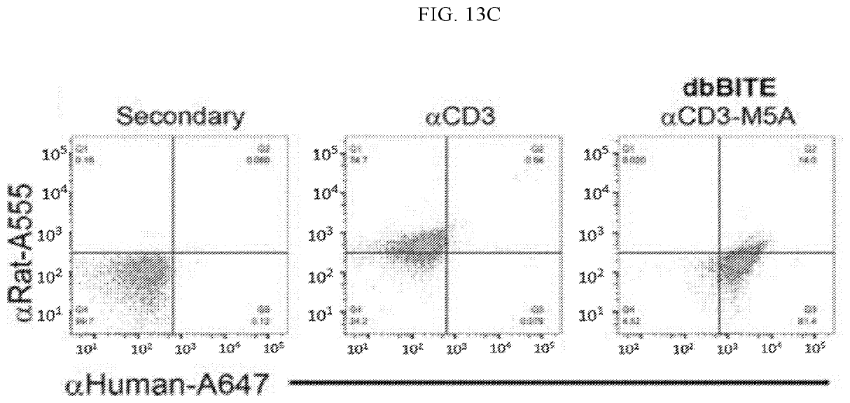

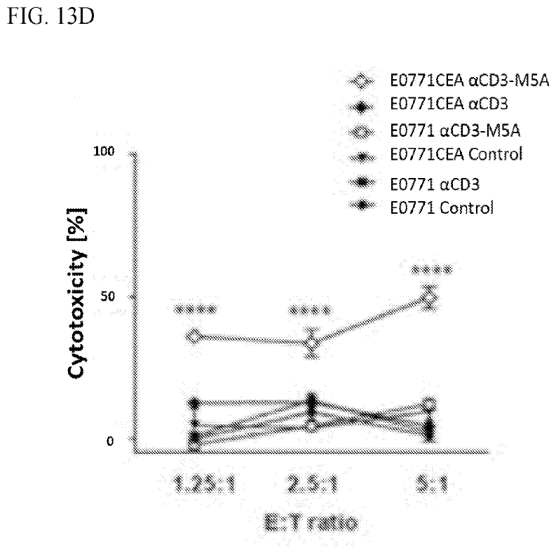

In order to test a biocompatible version of the dbBiTE in this model system, applicants generated a dbBiTE comprising rat anti-mouse CD3 and anti-CEA M5A. The in vitro cytotoxicity of this dbBiTE was first demonstrated with two CEA transfected cell lines, murine mammary carcinoma line E0177 and murine colon carcinoma line MC38, compared to untransfected parental cells. As illustrated in FIG. 13A, M5A binds only to the CEA+target with no binding of the rat secondary antibody, while the dbBiTE coated targets bind both secondary antibodies. Analogous experiments with murine colon carcinoma cell line MC38 with or without transfected CEA validate dbBiTE binding of both secondary antibodies, while M5A binds only to the CEA+target and not the secondary antibody (FIG. 13B). Similarly, anti-CD3 coated activated murine T-cells bind rat secondary antibody only, while dbBiTE coated activated murine T-cells bind both secondary antibodies (FIG. 13C). Further, the dbBiTE coated T-cells exhibited significant cytotoxicity to CEA+targets in comparison to CEA− controls as shown in FIG. 13D).

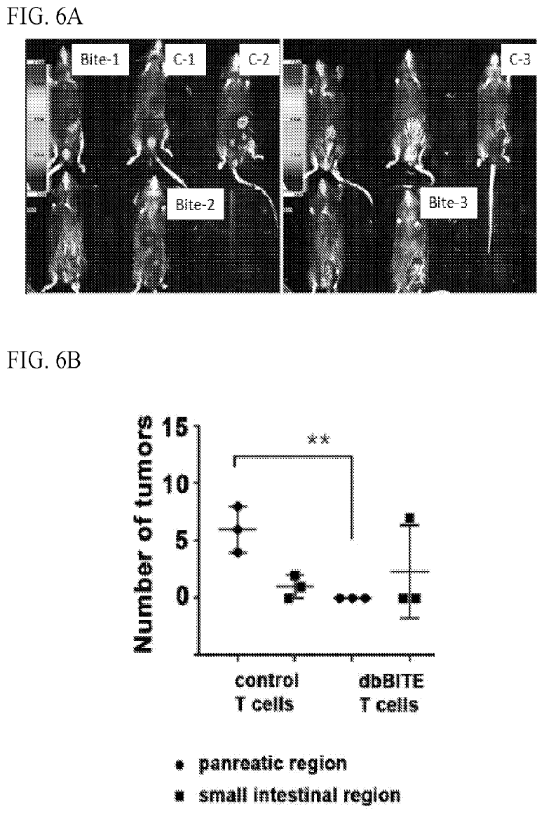

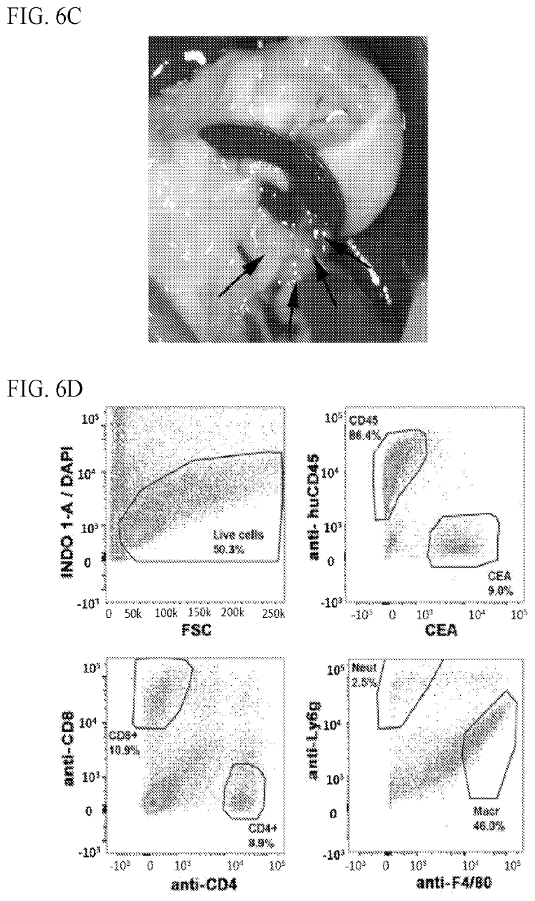

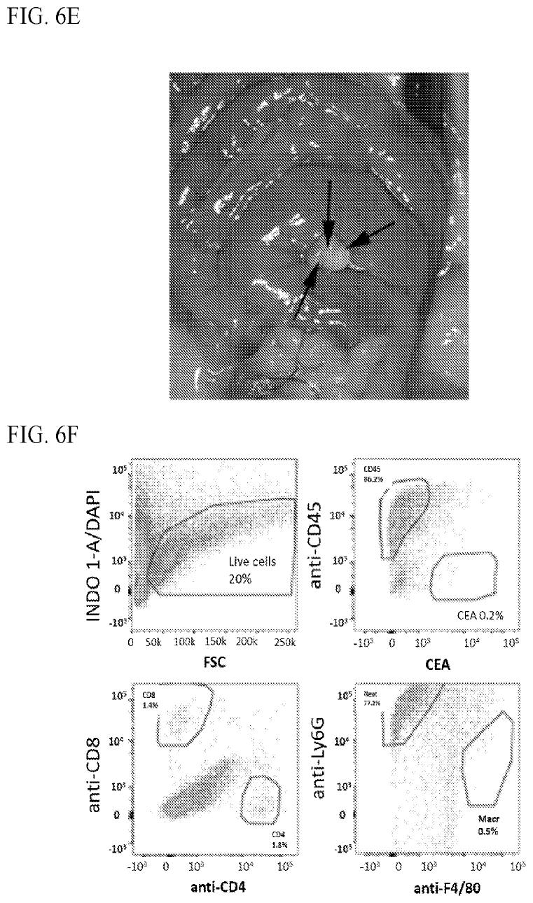

A limited therapy study of dbBiTE coated T-cells was performed in CEA transgenic mice bearing the syngeneic colon cancer cell line MC38 transfected with CEA and luciferase. Tumors were inoculated i.p. and 3 mice were selected as controls (little or no luciferase expression after 9 days) and 3 with carcinomatosis (FIG. 6A). The mice were treated i.p. four times every three days with 10M activated murine spleen T-cells either coated or not with 1 μg of dbBiTE. Four days after the last injection of dbBiTE coated T-cells, the mice were euthanized and examined for the presence of tumor nodules. The number of tumor nodules that were found either peri-pancreatic or periintestinal are shown in FIG. 6B along with micrographs shown in FIG. 6C and FIG. 6E. Tumor nodules were then digested and analyzed by flow for the presence of CEA, CD45 lymphocytes, CD4 or CD8 T-cells, and Ly6G neutrophils or F4-80 macrophages (FIG. 6D). The results demonstrate an absence of CEA in dbBiTE treated tumors and infiltration of Ly6G+ neutrophils, while CEA is present in controls along with infiltration of F4/80+ macrophages. This preliminary therapy study indicates that dbBiTE coated T-cells have therapeutic potential, and with further development, may become an alternative to the genetic manipulation necessary with CAR T-cell therapy.

Discussion

This study was initiated to extend the scope of BiTEs beyond the technology requiring genetically engineered dual scFvs, and beyond the approach of coating activated T-cells with a heterogenous mixture of bispecific antibodies. While genetically engineered BiTEs from dual scFv fragments to CD3 and either EpCAM or CD19 have shown significant anti-tumor activity in the clinic [8-10], it is important to extend this approach to readily available intact tumor specific antibodies. In this regard, CEA is an excellent target for many solid tumors including colon, breast, pancreas, and medullary thyroid carcinoma [11]. Although several groups have generated anti-CD3/anti-CEA dual scFv BiTEs [12, 13], their clinically utility is subject to several challenges common to all dual scFv BiTES. A major challenge is that the production of dual scFv BiTEs requires proper protein folding for subsequent large-scale production for clinical use. A second challenge is the need to continuously infuse these BiTEs for therapy due to their rapid blood clearance via the kidney. Since many humanized anti-tumor antibodies have become FDA approved therapeutics, it is highly desirable to generate bispecific antibodies directly from intact antibodies circumventing these two problems. First, there is no need to reengineer the bispecific BiTE, and if an intact anti-tumor IgG is FDA approved, one may obtain it from the pharmacy. Similarly, a murine anti-human CD3 antibody is commercially available and clinically tested [14]. Second, once combined into a bispecific format, their combined molecular weights (300 kda) would preclude blood clearance by the kidney. Although an attractive idea, many attempts to produce such antibodies retaining their dual specifities has been met with difficulty. Among the approaches, knobs-into-holes [15], DuoBody [16], and Cross-Mabs [17], all require re-engineering the parent antibodies.

In this study, applicants demonstrate that cross-linking of murine anti-human OKT3, an IgG2a, to a humanized anti-CEA IgG1 resulted in dbBiTE that was isolated in high yield and retained high binding to cells expressing human CD3 or CEA. Importantly, since both antibodies retain both antigen combining arms, they retain the property of avidity, inherent to all intact antibodies, a feature that is lost with dual scFv BiTEs. To emphasize this feature, applicants have named them dbBiTES for their dual specific, bivalent properties. Since biological products are expected to meet high standards in terms of molecular characterization, it was important to analyze the physical characteristics of dbBiTEs. EM characterization of the resultant 300 kDa particles revealed a flexible six-lobed structure with considerable density at the hinge region, consistent with our expected model. Indeed, the structural model generated using molecular docking and dynamics of the two IgG antibodies was consistent with the EM analysis. Finally, dbBiTE coated human or murine T-cells demonstrated significant and specific anti-CEA cytoxicity for CEA positive target cells. Thus, this approach may be exploited to generate antibody directed activated T cells without the need for genetic engineering, or alternatively, may be used to coat CAR-T cells to give them additional properties.

Since dbBiTEs are at an early stage of development, it's a task to demonstrate all of their advantages or to predict all of their disadvantages. The presence of intact Fc domains may be viewed as either an advantage or a disadvantage. One clue to their in vivo function was the difference in recruitment of myeloid cells to tumors treated in vivo. dbBiTE coated T-cells appeared to dramatically increase neutrophil infiltration, while in controls, macrophage infiltration dominated. Clearly, more model systems need to be tested to determine if this dichotomy can be generalized and if it truly reflects the Fc activity of dbBiTEs. Overall, results indicate powerful approach can be generalized, since it was demonstrated for both all-human and all-murine systems.

### Example 2: Materials and Methods for Cell Surface Antibody Click Chemistry to Generate Dual Specific Bivalent T-Cell Engagers

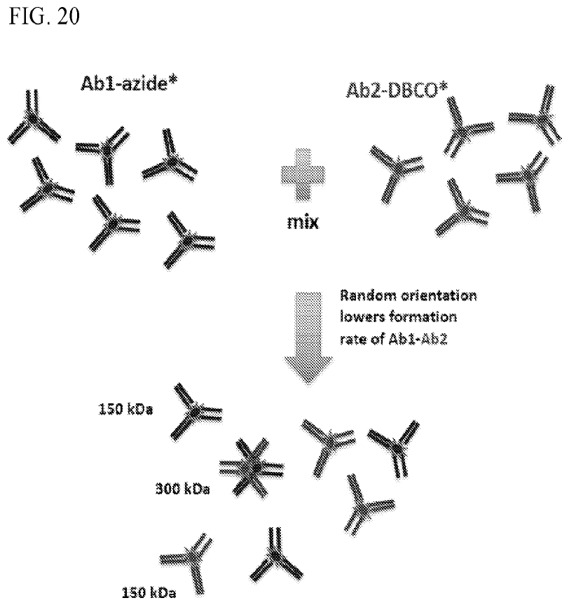

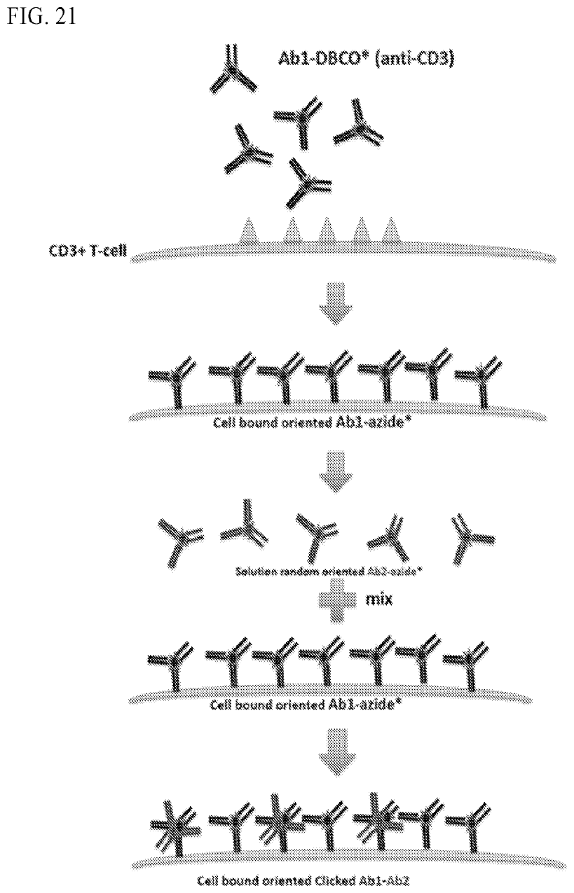

Applicants have shown two click chemistry group derivatized antibodies can react in solution to form bdBiTEs. However, the random orientation of the two derivatives antibodies in solution lowers the formation rate of the bdBiTEs as shown in FIG. 20. Further, the solution formed dbBiTEs requires purification of the intact bdBiTEs from any unreacted antibody in the reaction solution. Thus, applicants coated autologous immune cells with a click chemistry derivatized antibody, allowed the derivative antibody to bind to the immune cells, and incubated the coated immune cells with a second antibody derivatized with a complementary click chemistry group. The two complementary click chemistry groups reacted to form a hinge-region covalently conjugated bdBiTE as illustrated in FIG. 21.

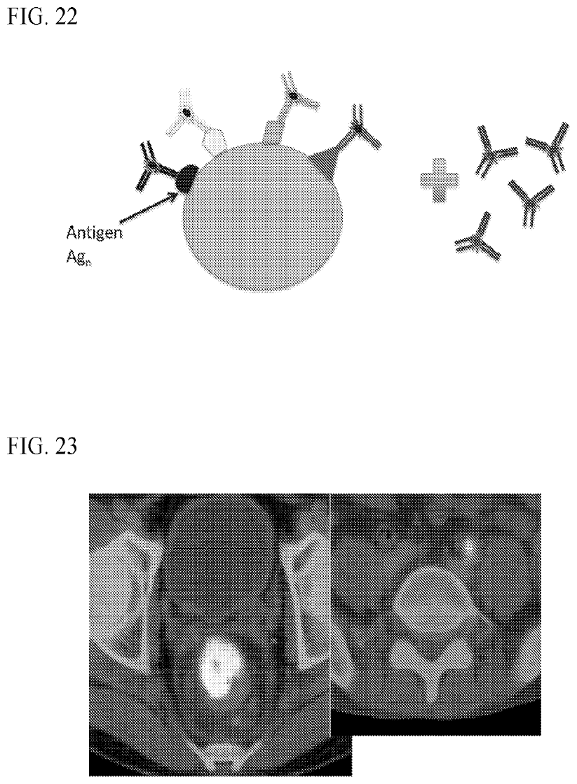

This approach allows homogenous formation of cell surface bdBiTEs without need for a further purification step. This approach also allowed faster formation of the bdBiTE due to the orientation of the first derivatized antibody on the surface of the immune cells. Further, this approach allows the possibility of multiplexing, with a variety of effector cell specific derivatized antibodies which can be clicked to a single or multiple target cell specific derivatized antibodies (FIG. 22).

Materials

Dibenzocyclooctyne-amine (BP-22066) and bromoacetamido-PEG₅-azide (BP-21801) were purchased from Broadpharm (San Diego, Calif.). Murine anti-human CD3 (OKT3; InVivoMAb, BE0001-2) was from BioXCell (Lebanon, N.H.) and rat anti-murine CD3 (Catalog Number: 100331, LEAF purified) was from Biolegend (San Diego, Calif.).

Synthesis of Bromoacetamido-DBCO

Bromoacetic anhydride (41.0 mg, 0.15 mmol) and NaHCO₃ (25.2 mg, 0.3 mmol) were added to dibenzocyclooctyne-amine (DBCO-NH₂; 27.52 mg, 0.1 mmol) in 400 μL dry DMF. After rotating at ambient temperature for 2 h under Argon, the mixture was purified by a Gemini C18 column (Phenomenex, CA. 4.6×250 mm) on an Agilent (1260 infinity) HPLC instrument using a mobile phase consisting of 0.1% TFA in H₂O (solvent A) and 0.1% TFA in acetonitrile (solvent B) with a linear gradient from 0% B to 95% B in 32 min at a flow rate of 1 mL/min. The product (18.7 mg; yield: 47.1%) was pooled and stored dried at 4° C. The mass of the product was confirmed by ESI mass spectrometry on a Thermo LTQ-FT; calculated (M+H+): 397.27, found: 397.06.

Reduction of Hinge Cysteines in an IgG Antibody

OKT-3: OKT-3 (2 mg, 13.3 nmol) in 234 μL PBS was reduced at 37° C. for 2 hours under Argon with a 30 molar excess of tris (2-carboxyethyl) phosphine (TCEP). TCEP was removed by desalting on a spin column (Zeba, 7 kDa MW cutoff, Thermo Scientific).

Humanized OKT-3: The humanized anti-CD3 mAb or humanized OKT-3 (hOKT-3) is a CDR grafted version of the murine OKT3 hybridoma named gOKT3-5 (Adair, J. R. et al, 1994 Hum Antibod Hybridomas). The genes encoding the gOKT3-7 variant, composed of the gLC and gHG, were synthesized, cloned into the pEE12/6 glutamine synthetase vector (Lonza Biologics, Cambridge, UK) and transiently expressed in EXPI293 cells (Thermo Fisher Scientific, Waltham, Mass.). The secreted antibody was purified by Protein A (Millipore Sigma, St Louis, Mo.) and ceramic hydroxyapatite chromatography (Bio-Rad Laboratories, Hercules, Calif.). hOKT-3 (2 mg, 13.3 nmol) in 294 μL PBS was reduced at 37° C. for 2 h under Argon with a 30 molar excess of TCEP. The TCEP was removed by desalting on a spin column (Zeba, 7 kDa MW cutoff, Thermo Scientific).

Alkylation of a Reduced Antibody with Bromoacetamido-DBCO

OKT-3: Reduced OKT-3 was reacted with a 20 fold molar excess of bromoacetamido-DBCO at RT overnight under Argon. Subsequently, excess bromoacetamido-DBCO was removed by dialyzing vs. 2 L of PBS with five buffer exchange steps. The conjugation of bromoacetamido-DBCO to OKT-3 was confirmed by mass spectrometry on an Agilent 6520 Q-TOF system. Results indicate there was DBCO per light chain and four DBCO per heavy chain.

Humanized OKT-3: Reduced-hOKT-3 was reacted with a 20 fold molar excess of bromoacetamido-DBCO at ambient temperature overnight under Argon. The excess bromoacetamido-DBCO was subsequently removed by dialyzing vs 2 L of PBS with five buffer exchange steps. The conjugation was confirmed by mass spectrometry on an Agilent 6520 QTOF. There was one DBCO per light chain and four DBCOs per heavy chain.

Alkylation of a Reduced Antibody with Bromoacetamido-PEG₅-Azide

Humanized anti-CEA antibody (M5A) (2 mg, 13.33 nmol) in 400 μL of PBS was reduced at 37° C. for 2 hr under Argon with a 30 molar excess of TCEP. The TCEP was removed by desalting on a spin column (Zeba, 7 KDa MW cutoff, Thermo Scientific). The reduced M5A was reacted with a 100 fold molar excess of bromoacetamide-PEG₅-N₃ at ambient temperature overnight under Argon. The excess bromoacetamide-PEG₅-N₃ was removed by dialyzing against 2 L of PBS with five buffer exchange steps. The conjugation was confirmed by mass spectrometry on an Agilent 6520 Q-TOF system. There was one PEG₅-N₃ per light chain of M5A and 3 per heavy chain of M5A.

A portion of the M5A-PEG₅-N₃ was reacted with a 20 molar excess of Alexa-488-NHS (Catalog Number: A20000, Thermo Fisher) at pH 7.5 in ambient temperature for 2 hr under Argon. The excess Alexa-488 was removed by dialyzing against 2 L of PBS with five buffer exchange steps. The conjugation was confirmed by mass spectrometry on an Agilent 6520 Q-TOF system and by size exclusion column chromatography (SEC) (Superdex 200, 10×300 GL, GE Healthcare) equipped with a fluorescent detector (Ex/Em: 494/517 nm, JASCO FP-2020 Plus) at a flow rate of 0.5 mL/min in PBS using a GE AKTA Purifier FPLC System.

Solution Based Click Chemistry of Dual Specific Bivalent BiTEs and Purification by SEC HPLC

OKT-3-DBCO (2 mg, 13.33 nmol) in 360 μL PBS was added to M5A-PEG₅-N₃ (2 mg, 13.3 nmol) in 340 μL PBS, pH 7.25. The mixture was rotated at ambient temperature for 1 hr under Argon, and incubated at 4° C. overnight. The antibodies, covalently conjugated by the click chemistry reaction, were purified by SEC (Superdex 200, 10×300 GL, GE Healthcare) at a flow rate of 0.5 mL/min in PBS using a GE AKTA Purifier FPLC System (FIG. 2A). Two peaks were collected and concentrated using a 2 mL Vivaspin 10 MWCO (Sartorius, UK). The molecular sizes of Peak 1 (300 kDa) and Peak 2 (150 kDa) were determined by SDS gel electrophoresis (NuPAGE 4-12% Bis-Tris Gel; Life Technologies, CA) under non-reducing conditions along with antibody standards.

Production of Activated Human T-Cells

Human PBMC from discard blood (no consent required) were isolated by centrifugation of whole blood in Ficoll-Paque PLUS density gradient media (GE Healthcare) for 30 minutes (500 g) and washed with PBS three times. After counting and checking cell viability, PBMC were plated on a 6 well plate which was previously coated with anti-CD3 antibody (2 μg/mL of OKT-3 for 2 hr at 37° C.), washed with PBS, and cultured at a concentration 2×10⁶ cells/mL in RPMI1640 containing 10% FBS and 100 U/ml of recombinant human IL-2 (BioLegend, San Diego, Calif.). After 72 h, cultures of activated cells were expanded by platting 5×10⁵ cells/mL in RPMI 1640 media containing 10% FBS and 100 U/ml of recombinant human IL-2. The cells were cultured for 5-7 days and used for in vitro functional experiments. Following the culturing step, over 80% of cells were CD3 positive.

Cell Based Click Chemistry of Dual Specific Bivalent BiTEs

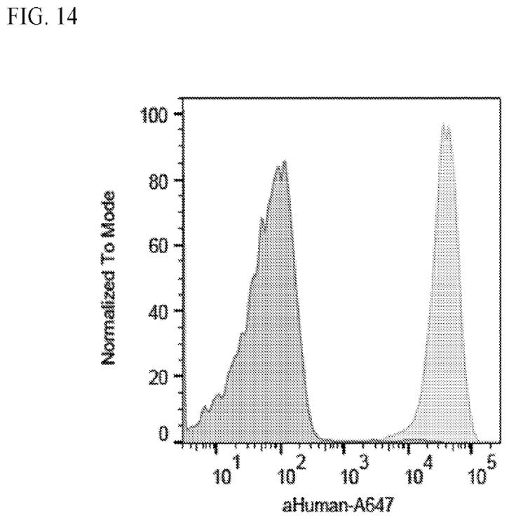

Humanized OKT-3: 1 to 2 μg/mL of hOKT-3-DBCO in PBS with 0.5% BSA was incubated on ice for 30 minutes with 1-2 mL of human T-cells at a concentration of 10×10⁶ cells/mL, The amount of hOKT3 bound to the T-cells was evaluated by staining with Alexa-647 conjugated goat anti-human IgG on a LSRFortesa X-20, BD Biosciences Flow analyzer. Results shown in FIG. 14 indicate that hOKT3 was successfully bound to the surface of the activated human T-cells.

The hOKT3-DBCO coated T-cells were rinsed two times with cold PBS to remove excess unbound antibody. The hOKT3-DBCO coated T-cells were then incubated with Alexa 488 (A488) conjugated anti-CEA (M5A-PEG₅-N₃) on ice for 2 hours to allow the click chemistry reaction to occur at the surface of the T-cells. The click chemistry reaction was evaluated with 0, 5.0, 10.0 and 20.0 μg/mL of Alexa-488 conjugated M5A-PEG₅-N₃. Following incubation of the hOKT3-DBCO coated T-cells with the various concentrations of Alexa-488 conjugated M5A-PEG₅-N₃, the cells were rinsed with cold PBS two times to remove unreacted antibody. The amount of cell surface bispecific click chemistry conjugated antibody was assessed by flow cytometry using the Alexa-488 channel. Results illustrated in FIG. 15 show formation of bispecific antibody due to the click chemistry reaction between hOKT3-DBCO and Alexa-488 conjugated M5A-PEG₅-N₃ at the T-cell surface.

Redirected Cytoxicity of Activated Human T-Cells Bearing Cell Surface-Clicked Dual Specific Bivalent BiTEs

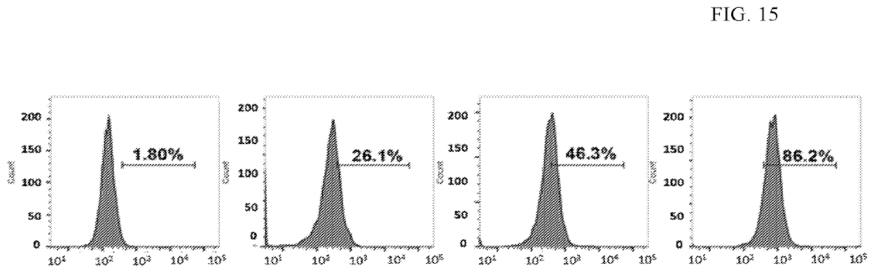

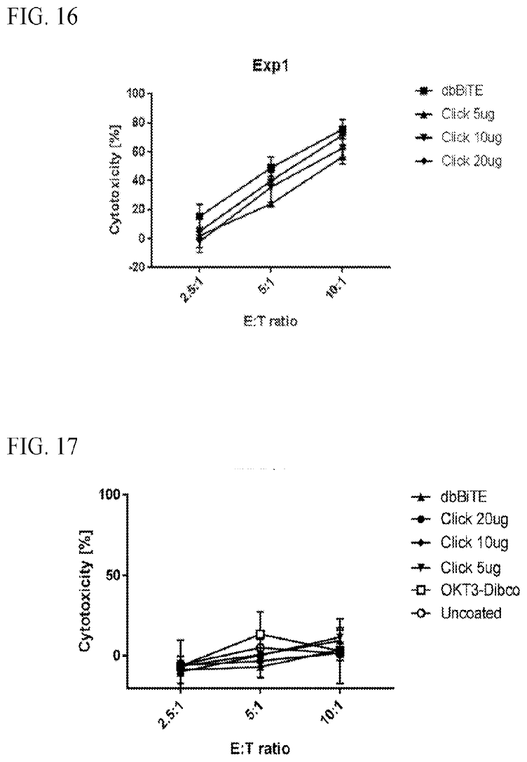

The cell surface clicked dbBiTE bound to T-cells were assessed for their ability to kill CEA positive target cells. CEA+MD-MB231 target cells expressing GFP were plated on 96-well plates at 10×10³ cells per well in 100 μL of Dulbecco's Modified Eagle Medium (DMEM) containing 10% FBS. Following an 18 hour incubation, 100 μL of dbBiTE coated activated T-cells were added to target cells in concentrations corresponding to final effector (E) to target (T) ratios of 10:1, 5:1, and 2.5:1. Following a 24 hr incubation, cytotoxicity was measured by the decrease of GFP expressing cells. As illustrated in FIG. 16, the cell surface clicked dbBiTE coated cells exhibited similar cytotoxicity in comparison to solution generated dbBiTE coated cells, and showed a dose dependent cytotoxic response over the E:T ratios tested.

The cell surface clicked dbBiTE coated T-cells were incubated for 24 hours with the CEA negative human breast cell line MDA-MB-231 expressing GFP. Results illustrated in FIG. 17 show that the activated T-cells with cell surface clicked dbBiTEs did not kill CEA negative cells, indicating the cell surface clicked antibodies display high specificity for their target.

Release of IFNγ by Activated Human T-Cells Bearing Cell Surface-Clicked Dual Specific Bivalent BiTE Engaging Target Cells.

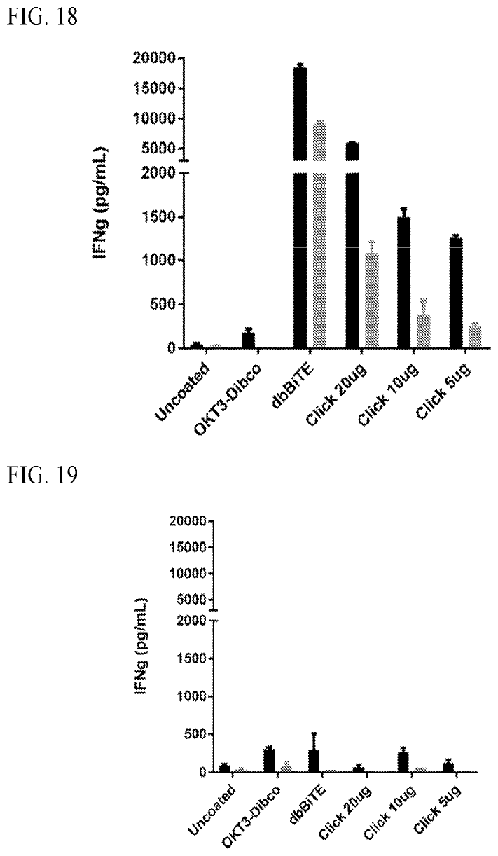

Release of IFNγ is considered a functional measure of activated T-cells that have actively recognized their specific target cells. To evaluate cell surface-clicked dbBiTE coated T-cell recognition of CEA positive cells, cell supernatants were collected and IFNγ production was measured by enzyme immunoassay (EIA). hOKT3-Dibco coated cells were treated with either 5, 10, or 20 μg/mL of M5A-PEG₅-N₃ for the cell surface formation of dbBiTE by click chemistry. The surface coated dbBiTE activated T-cells were then incubated with CEA positive target cells at E:T ratios of 10:1 and 5:1. The results shown in FIG. 18 demonstrate maximal release of IFNγ obtained with hOKT3-Dibco coated cells treated with 20 μg/mL of M5A-PEG₅-N₃ at an E:T of 10:1. This result was roughly equivalent to the positive control of pre-formed dbBiTE coated T-cells at an E:T of 5:1. The background level of hOKT3 coated T-cells, which were not treated with M5A-PEG₅-N₃, was less than 100 μg/mL IFNγ in comparison to greater than 200 μg/mL IFNγ for hOKT3-Dibco coated cells treated with 5 or 10 μg/mL of M5A-PEG₅-N₃, respectively. Results shown in FIG. 19 indicate minimal IFNγ release when bispecific antibody coated T-cells were incubated with CEA negative cells. These results further validate specific recognition and cytotoxicity of surface-clicked dbBiTE coated T-cells for target cells.

### Example 3: Dual Specific Bivalent Bite Coated Cells with Immunopet Targeting

Applicants will develop novel cell-based therapies for CRCC. ImmunoPET targeting of two commonly expressed antigens, CEA and TAG72 will guide the selection for appropriate targeted therapy. The three aims are as followed: the first employing immunoPET with Cu-64 labeled anti-CEA and anti-CC49 (TAG72) antibodies, the second, development of single and combined dbBITE and CAR T directed therapies, and the third obtaining data using immunoPET and monitoring of response to mono versus dual T-cell directed therapies.

While colorectal cancer carries a good prognosis, 37% still die from metastatic disease (1). Death from colorectal cancer is predominantly due to liver failure or widespread peritoneal metastasis (CRCC). Little progress has been made in diagnosing and treating CRCC. Once CRCC is identified by conventional imaging, combination chemotherapy is ineffective (2). Quality of life is compromised due to frequent hospitalizations for bowel obstruction, obstructive nephropathy and/or malignant ascites. Death occurs rapidly with a median survival of 6 months (3).

There are two main reasons for dismal outcomes in CRCC: (i) Delay in diagnosis; and (ii) Lack of effective therapies. This project will provide significant data on the use of tumor-targeted immuoPET in CRCC patients currently seen in the clinic. Based on these data, future studies could be designed to immunoPET image newly diagnosed colorectal patients to determine the presence or absence of CRCC. In terms of the second reason, numerous studies have demonstrated that the best oncologic outcomes for regional therapies are in patients that have limited CRCC burden (4). However, conventional imaging (Multi-detector CT or Dynamic contrast-enhanced MRI) has poor sensitivity (44-63%) to detect CRCC (5). As a result, many patients have advanced disease at surgical exploration and curative-intent surgery is not an option. For patients that have limited CRCC burden at surgical exploration, a highly aggressive approach of removing all macroscopic tumors (cytoreductive surgery or CRS) followed by heated intraperitoneal chemotherapy (HIPEC) at the same time as CRS, has been investigated. Even in highly selected patients, this approach carries significant morbidity and mortality with limited efficacy (6). Complimentary diagnostic and therapeutic approaches are urgently needed. Applicants propose an immunoPET biomarker-driven approach to identify candidates for potent target-specific, cell-based therapies that are directly introduced into the peritoneal cavity, bypassing the need for systemic delivery, and likely reducing off-target effects. Cell-based therapies guided by target specific immunoPET will greatly aid in the selection of patients and monitoring the response to therapy

Applicants will combine CAR T and BITE cell based therapies using a novel version of BITEs termed dbBITEs because they have not only dual specificities, but also are bivalent for each specificity, thus increasing their potency. Furthermore, their direct delivery into the peritoneal cavity is novel and expected to be more effective than systemic administration where cell-based therapeutics tend to accumulate in the spleen and liver. Data suggests that both approaches have significant activity in a CRCC model, but because of tumor regrowth, it is likely that neither approach alone will be sufficient.

ImmunoPET studies. The ability to directly image tumors with radiolabeled anti-tumor antibodies is beginning to demonstrate utility in the clinic, especially in the case of antibodies labeled with positron emitting radionuclides, allowing sensitive, quantitative PET imaging, often surpassing the results obtained by standard FGD PET imaging where only active metabolizing tumors are detected (20). In the case of tumor-targeted therapies such as BiTE or CAR T cells, immunoPET has the potential advantages of patient selection and monitoring the expression of the specific tumor target over the course of therapy.

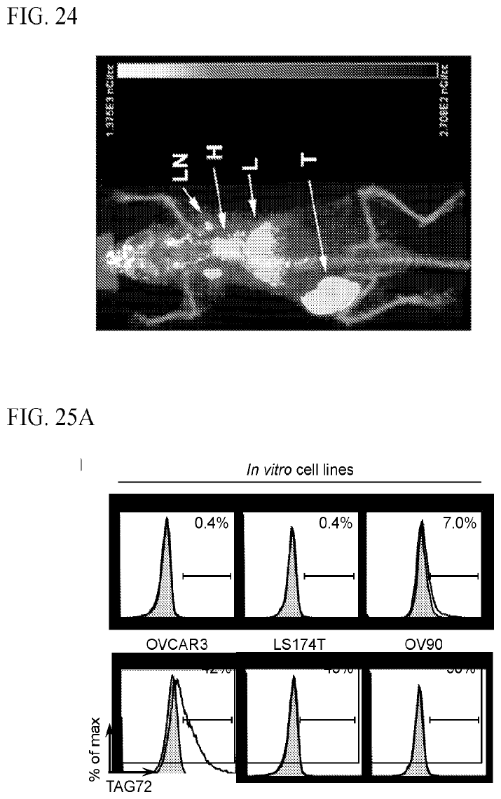

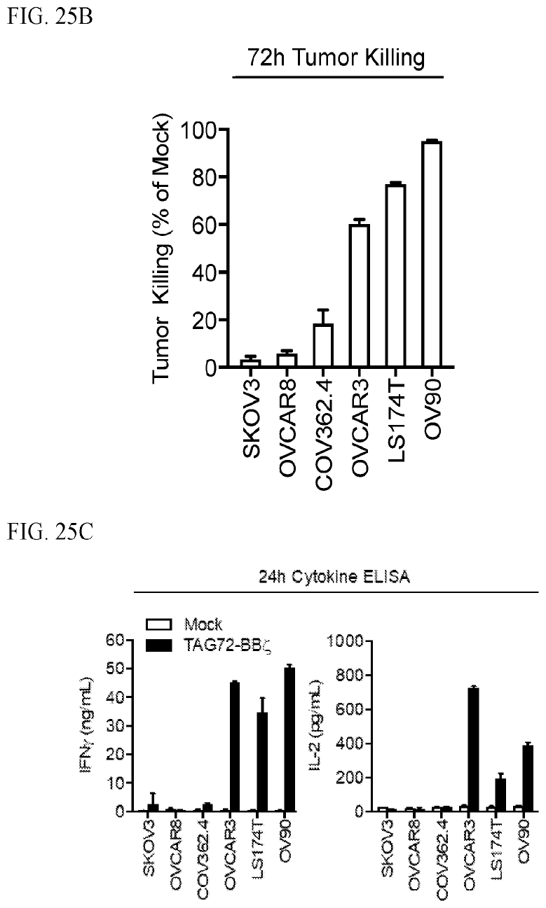

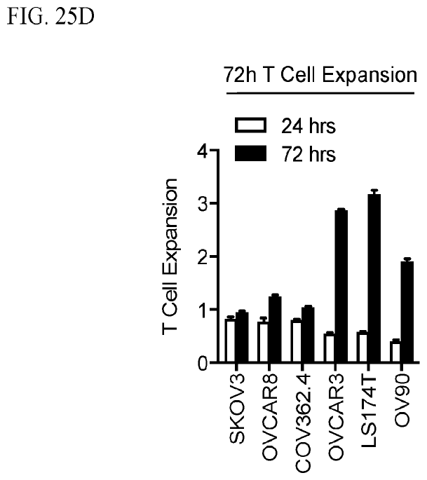

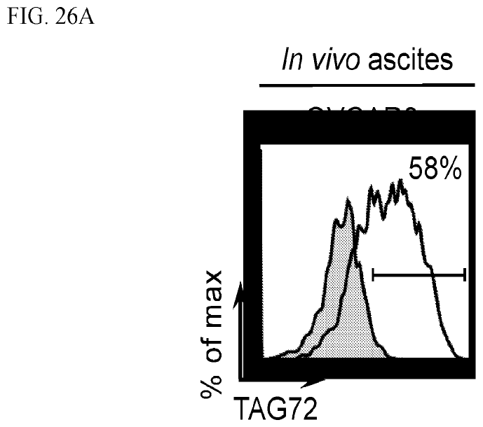

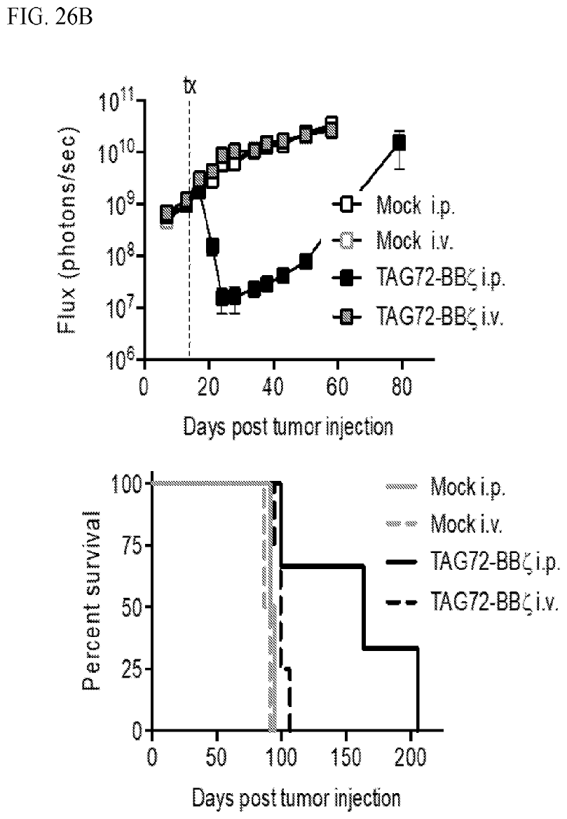

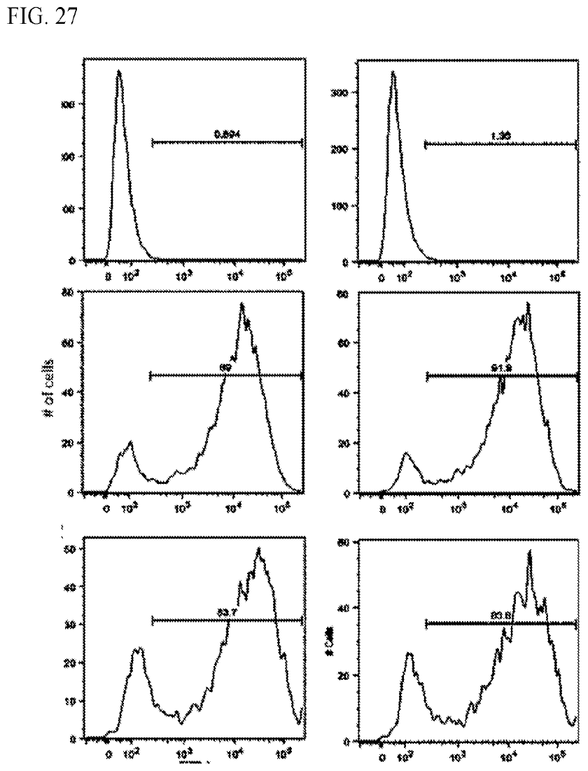

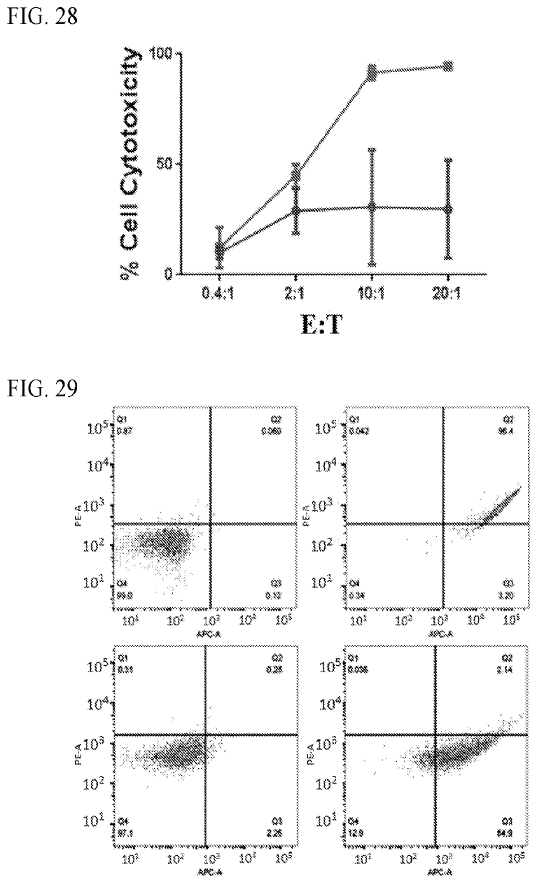

CEA is an attractive tumor target for colorectal ca where >90% of these tumors and their metastases express CEA (22). An example of imaging a rectal tumor and lymph node is shown in FIG. 23, which was not shown in standard Fludeoxyglucose (¹⁸F) (FDG) imaging. Tag72 is another attractive tumor target antigen expressed in >90% of colorectal and other solid malignancies (24). Immunoimaging and therapy with anti-TAG-72 antibody CC49 has been extensively performed in the clinic (25), demonstrating high target specificity. Applicants have produced multi-mg quantities of a humanized version of this antibody that was conjugated to DOTA, radiolabeled with Cu-64, shown to retain 100% immunoreactivity, and imaged TAG72 positive tumors in a preclinical model (FIG. 24).

Dual bivalent BITES (dbBITES). Applicants have generated bispecific antibodies by cross-linking two whole antibodies together at their hinge regions using Click chemistry. OKT3, a murine anti-human CD3 antibody widely used in the clinic (29) was conjugated to an anti-CEA M5A antibody. Since OKT3 is a murine antibody and M5A a humanized antibody, applicants were able to use anti-mouse and anti-human secondary antibodies to demonstrate that both specificities were present in the dbBITE.

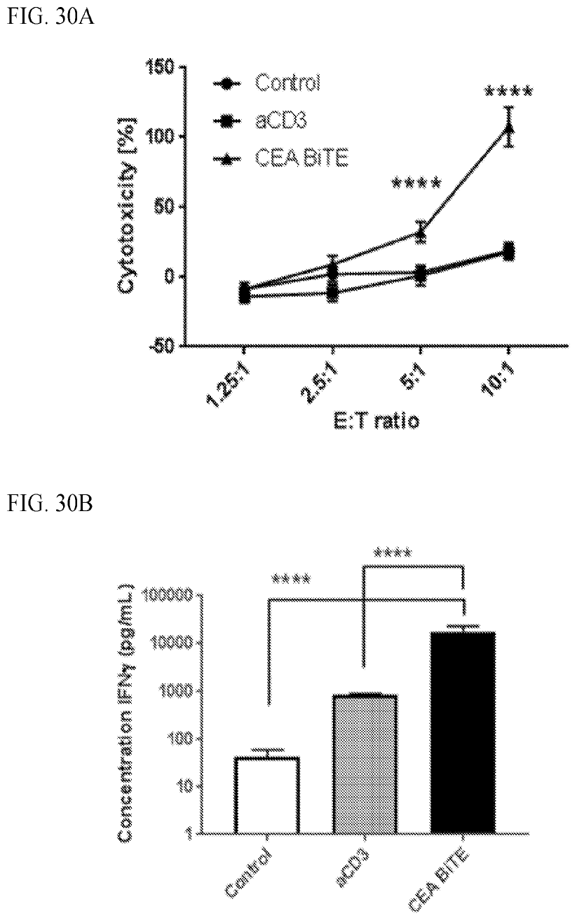

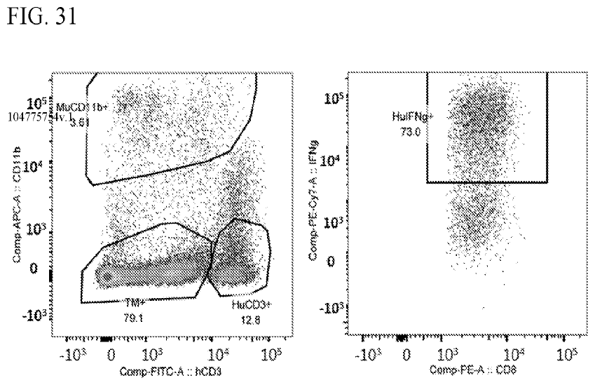

Both anti-CD3+IL2 activated human PBMCs and CEA+LS174T cells incubated with dbBITE stained positive with anti-mouse IgG-A555 and anti-human IgG-A647 demonstrating that both activities were present (FIG. 29). Cell killing was demonstrated by incubating PBMCs coated with dbBITE (1 ug/10⁶ cells) with LS174T targets at the indicated E:T ratios. Significant killing above controls was observed at E:T ratios of 5:1 and 10:1 (FIGS. 30A-B) that correlated with production of IFNγ (FIG. 30B). LS174T cells implanted I.P. in NOD/SCID mice and treated 4× with 1×10⁶ dbBITE coated human PBMCs resulted in significant infiltration of dbBITE/PBMCs (FIG. 31) into tumor nodules comprising necrotic tumor cells (data not shown).

Applicants also generated an OKT3/CC49 dbBITE that performed similarly to the OKT3/M5A dbBITE on SE HPLC with a yield of about 50%.

Rationale. In order to improve outcomes for the treatment of CRCC, applicants propose a tumor-targeted approach combining diagnosis with immunoPET and cell-based therapy based on the same agents. Since it is unlikely that a single targeted approach will succeed, two target antigens, CEA and TAG72, were selected, both with ample clinical significance in colorectal cancer. Applicants will start by performing immunoPET imaging on CRCC patients to determine the value of immunoPET versus standard imaging using surgical resection as the gold standard for both techniques. At the same time, applicants will develop the cell-based therapeutics, in order that both immunoPET and the cell based therapies can be combined. Although radiolabeled antibodies to CEA and TAG72 have been shown to have exquisite specificity in terms of imaging tumors over normal background and normal expression of the two antigens, these (naked) antibodies have little or no therapeutic activity. Although these antibodies have therapeutic activity when conjugated to drugs or therapeutic isotopes, their therapeutic efficacy as single agents in either format is poor. On the other hand, antibody directed cell therapies such as CAR T have shown encouraging results in hard to treat malignancies. Thus, applicants propose to develop a study wherein CRCC malignancies are selected by pre-therapy immunoPET, treated with antibody directed cell therapies, and followed by immunoPET to determine efficacy. This approach is predicated on: (i) radioimmunoimaging trials with anti-CEA antibodies, including Cu-64/DOTA-antibody PET imaging, (ii) the production of clinical grade antibodies and autologous CAR-T cells, and (iii) the development of a new BITE format that allows killing of tumor cells with dbBITE coated autologous T-cells.

Evaluate the efficacy of engineered T-cell therapies in pre-clinical models of CRCC. Eight pre-clinical animal studies will be performed. (i) All-murine CEA CAR T cells in an immunocompetent model using CEA Tg mice. (ii) Humanized CEA CAR T cells in the LS174T immunocompromised model. (iii) Humanized TAG72 CAR-T cells in the LS174T immunocompromised model. (iv) All murine CEA dbBITES coated T-cells in an immunocompetent model using CEA Tg mice. (v) CEA humanized dbBITES in the LS174T immunocompromised model. (vi) TAG72 dbBITES in the LS174T immunocompromised model. (vii) CEA CAR T cells coated with TAG72 dbBITES, and (viii) TAG72 CAR-Ts coated with CEA dbBITES.

All murine CEA dbBITES coated T-cells in an immunocompetent model using CEA Tg mice. The generation and functional characterization of anti-CEA×anti-CD3 (OKT3) dbBITES is described herein. While the short-term cytoxicity of dbBITE coated murine or human CD8 T-cells is similar, unlike CAR T cells, these cells do not expand in culture. Thus, while single infusions of CAR T cells may also expand in vivo and may require a single therapeutic infusion, dbBITE coated T-cells exert their killing over a period of a few days only. An advantage of acute versus chronic immune-cell-basecytotoxicity is less overall toxicity, a potential problem with long lasting CAR T cells that still express their endogenous T cell receptors. Using conditions for activation and expansion of murine T-cells described herein, applicants obtain a 70/30 mixture of CD8/CD4 T-cells with <1% contamination of B-cells and CD11b+ myeloid cells.

Groups of 8 mice with MC38±CEA CRCC will be treated weekly for four weeks with 5×10⁶ or 10×10⁶ all-murine dbBITE coated murine T-cells (mix of CD8s and CD4s; 32 mice). Controls will be PBS untreated and anti-CD3 activated T-cells (32 mice) for a subtotal of 64 mice. Tumor growth will be monitored weekly by bioluminescence and survival by Kaplan-Meier curves. Reduction of tumor growth (total bioluminescence) by >50% or >50% increased survival will be considered significant. This study will be repeated at a single dose (48 mice) for a total of 112 mice. In the repeat, the mice will be euthanized after 4-6 weeks of therapy (to be determined based on first study) for collection of blood and tissues for analysis of lymphocyte subsets (CD4, CD8, CD11b, CD62L, CCR7, CD95, IFNγ, FoxP3, PD-1, CTLA4, TIM-3) by FACS.

CEA humanized dbBITES in the LS174T immunocompromised model. Humanized anti-CEA antibody M5A conjugated to OKT3 is coated on purified, activated human T-cells isolated from PBMCs from discard blood. Cytoxicity against CEA+LS174T target cells has already been performed. Groups of 8 mice (NOD/SCID) bearing LS174T/luc CRCC (>50% take on IP injection of 200,000 cells) will be treated four times with 5×10⁶ or 10×10⁶ dbBITE coated T-cells (16 mice). Controls will be PBS untreated and anti-CD3 coated T-cells (16 mice) for a sub total of 32 mice. The repeat will be performed with one dose (24 mice) for a total of 56 mice, allowing direct comparison of the efficacy of therapy against the same CRCC tumors for anti-CEA vs anti-TAG72 CAR Tcells.

TAG72 dbBITES in the LS174T immunocompromised model. Anti-CC49 antibody was conjugated to OKT3. TAG72 dbBITE coated human PBMCs exhibited similar killing of LS174T cells compared to the anti-CEA×OKT3 dbBITE coated human PBMCs. Further characterization is underway, including EM analysis, serum stability, and cytotoxicity against TAG72neg targets.

Groups of 8 mice with LS174T CRCC will be treated weekly for four weeks with 5×10⁶ or 10×10⁶ all-human dbBITE coated human T-cells. Controls will be PBS untreated and anti-CD3 activated T-cells. Tumor growth will be monitored weekly by bioluminescence and survival by Kaplan-Meier curves. Reduction of tumor growth (total bioluminescence) by >50% or >50% increased survival will be considered significant. The number of mice will be 8 per group×2 doses×1 cell type+2 controls for a subtotal of 32 mice. This study will be repeated for one dose (24) mice for a total of 56 mice. In the repeat, the mice will be euthanized after 4-6 weeks of therapy for collection of blood and tissues for analysis of lymphocyte subsets (CD4, CD8, CD11b, CD62L, CCR7, CD95, IFNγ, FoxP3, PD-1, CTLA4, TIM-3) by FACS.

CEA CAR T cells coated with TAG72 dbBITES. Dual antigen cell based therapy will be performed by coating CEA CAR T cells with TAG72 dbBITEs, or by coating TAG72 CAR T cells with CEA dbBITES. This study begins with in vitro testing of CEA CAR T cells coated with TAG72 dbBITEs. Cytotoxicity of these cells versus uncoated CEA CAR T cells or TAG72 dbBITE coated T-cells will be performed using lucLS174T cells as targets. Possible outcomes include, equivalent, worse or better killing based on E:T ratios. In all cases, applicants will compare the results to a mixture of CEA and TAG72 CAR T cells, or a mixture of CEA and TAG72 dbBITE coated T-cells. The best killing results will go forward to animal studies. A typical animal study is shown below.

Groups of 8 mice with LS174T CRCC will be treated weekly for four weeks with 5×10⁶ or 10×10⁶ all-human dbBITE coated CEA CAR T cells (total of 16 mice in this study). Controls will be PBS untreated and anti-CD3 activated T-cells (16 mice). Tumor growth will be monitored weekly by bioluminescence and survival by Kaplan-Meier curves. Reduction of tumor growth (total bioluminescence) by >50% or >50% increased survival will be considered significant. This study will be repeated (24 mice) for a total of 56 mice. In the repeated study, the mice will be euthanized after 4-6 weeks of therapy for collection of blood and tissues for analysis of lymphocyte subsets (CD4, CD8, CD11b, CD62L, CCR7, CD95, IFNγ, FoxP3, PD-1, CTLA4, TIM-3) by FACS.

TAG72 CAR T cells coated with CEA dbBITES. This is the parallel study to the one above and will proceed in the same manner with a total 56 mice. Although the studies may lead to equivalent results due to their symmetric design, it is entirely possible that one or the other will yield superior results either in the in vitro, in vivo, or in both phases.

The overall goals of this project are to (1) establish immunoPET imaging for CEA and TAG72 to detect the antigens in subjects and to monitor the efficacy of the cell-based therapies and to (2) develop the cell based therapies, which include dbBiTE coated CAR T cells or dbBiTE coated Tcells using the two antigens as targets. The rationale is to justify the detection of these two prevalent antigens in CRCC and to select the best single agent(s) and best dual therapy agent(s). The choice will depend on which single agent cell therapy is used, TAG72 or CEA based. For dual targeted cell based therapy, applicants will use the ImmunoPET agent that performs best. Secondary goals of the proposed trial will be to determine the response to the cell-based therapy, presence or absence of antigen in surgical specimens, and presence or absence of T-cell infiltrates in surgical specimens.

### REFERENCES

1. Baeuerle P A, Reinhardt C: Bispecific T-cell engaging antibodies for cancer therapy. Cancer Res 2009, 69(12):4941-4944.
2. Klinger M, Benjamin J, Kischel R, Stienen S, Zugmaier G: Harnessing T cells to fight cancer with BiTE® antibody constructs—past developments and future directions. Immunol Rev 2016, 270(1):193-208.
3. Chan J K, Hamilton C A, Cheung M K, Karimi M, Baker J, Gall J M, Schulz S, Thorne S H, Teng N N, Contag C H et al: Enhanced killing of primary ovarian cancer by retargeting autologous cytokine-induced killer cells with bispecific antibodies: a preclinical study. Clin Cancer Res 2006, 12(6):1859-1867.
4. Ortho Multicenter Transplant Study G: A randomized clinical trial of OKT3 monoclonal antibody for acute rejection of cadaveric renal transplants. N Engl J Med 1985, 313(6):337-342.
5. Yazaki P J, Sherman M A, Shively J E, Ikle D, Williams L E, Wong J Y, Colcher D, Wu A M, Raubitschek A A: Humanization of the anti-CEA T84.66 antibody based on crystal structure data. Protein Eng Des Sel 2004, 17(5):481-489.
6. Li L, Bading J, Yazaki P J, Ahuja A H, Crow D, Colcher D, Williams L E, Wong J Y, Raubitschek A, Shively J E: A versatile bifunctional chelate for radiolabeling humanized anti-CEA antibody with In-111 and Cu-64 at either thiol or amino groups: PET imaging of CEA-positive tumors with whole antibodies. Bioconjug Chem 2008, 19(1):89-96.
7. Clarke P, Mann J, Simpson J F, Rickard-Dickson K, Primus F J: Mice transgenic for human carcinoembryonic antigen as a model for immunotherapy. Cancer Res 1998, 58(7):1469-1477.
8. Weidle U H, Kontermann R E, Brinkmann U: Tumor-antigen-binding bispecific antibodies for cancer treatment. Semin Oncol 2014, 41(5):653-660.
9. Dillon M, Yin Y, Zhou J, McCarty L, Ellerman D, Slaga D, Junttila T T, Han G, Sandoval W, Ovacik M A et al: Efficient production of bispecific IgG of different isotypes and species of origin in single mammalian cells. MAbs 2017, 9(2):213-230.
10. Brinkmann U, Kontermann R E: The making of bispecific antibodies. MAbs 2017, 9(2):182-212.
11. Hammarstrom S: The carcinoembryonic antigen (CEA) family: structures, suggested functions and expression in normal and malignant tissues. Semin Cancer Biol 1999, 9(2):67-81.
12. Osada T, Patel S P, Hammond S A, Osada K, Morse M A, Lyerly H K: CEA/CD3-bispecific T cell-engaging (BiTE) antibody-mediated T lymphocyte cytotoxicity maximized by inhibition of both PD1 and PD-L1. Cancer Immunol Immunother 2015, 64(6):677-688.
13. Oberst M D, Fuhrmann S, Mulgrew K, Amann M, Cheng L, Lutterbuese P, Richman L, Coats S, Baeuerle P A, Hammond S A: CEA/CD3 bispecific antibody MEDI-565/AMG 211 activation of T cells and subsequent killing of human tumors is independent of mutations commonly found in colorectal adenocarcinomas. MAbs 2014, 6(6):1571-1584.
14. Herve P, Flesch M, Cahn J Y, Racadot E, Plouvier E, Lamy B, Rozenbaum A, Noir A, Des Floris R L, Peters A: Removal of marrow T cells with OKT3-OKT11 monoclonal antibodies and complement to prevent acute graft-versus-host disease. A pilot study in ten patients. Transplantation 1985, 39(2):138-143.
15. Ridgway J B, Presta L G, Carter P: ‘Knobs-into-holes’ engineering of antibody CH3 domains for heavy chain heterodimerization. Protein Eng 1996, 9(7):617-621.
16. Spiess C, Zhai Q, Carter P J: Alternative molecular formats and therapeutic applications for bispecific antibodies. Mol Immunol 2015, 67(2 Pt A):95-106.
17. Labrijn A F, Meesters J I, de Goeij B E, van den Bremer E T, Neijssen J, van Kampen M D, Strumane K, Verploegen S, Kundu A, Gramer M J et al: Efficient generation of stable bispecific IgG1 by controlled Fab-arm exchange. Proc Natl Acad Sci USA 2013, 110(13):5145-5150.
18. Patterson J T, Gros E, Zhou H, Atassi G, Kerwin L, Carmody L, Zhu T, Jones B, Fu Y, Kaufmann G F: Chemically generated IgG2 bispecific antibodies through disulfide bridging. Bioorg Med Chem Lett 2017, 27(16):3647-3652.
19. Suloway C, Pulokas J, Fellmann D, Cheng A, Guerra F, Quispe J, Stagg S, Potter C S, Carragher B: Automated molecular microscopy: the new Leginon system. J Struct Biol 2005, 151(1):41-60.
20. Lander G C, Stagg S M, Voss N R, Cheng A, Fellmann D, Pulokas J, Yoshioka C, Irving C, Mulder A, Lau P W et al: Appion: an integrated, database-driven pipeline to facilitate EM image processing. J Struct Biol 2009, 166(1):95-102.
21. Rohou A, Grigorieff N: CTFFIND4: Fast and accurate defocus estimation from electron micrographs. J Struct Biol 2015, 192(2):216-221.
22. Sorzano C O, Marabini R, Velazquez-Muriel J, Bilbao-Castro J R, Scheres S H, Carazo J M, Pascual-Montano A: XMIPP: a new generation of an open-source image processing package for electron microscopy. J Struct Biol 2004, 148(2):194-204.
23. Voss N R, Yoshioka C K, Radermacher M, Potter C S, Carragher B: DoG Picker and TiltPicker: software tools to facilitate particle selection in single particle electron microscopy. J Struct Biol 2009, 166(2):205-213.
24. Radermacher M, Wagenknecht T, Verschoor A, Frank J: Three-dimensional reconstruction from a single-exposure, random conical tilt series applied to the 50S ribosomal subunit of Escherichia coli. J Microsc 1987, 146(Pt 2):113-136.
25. Goddard T D, Huang C C, Ferrin T E: Visualizing density maps with UCSF Chimera. J Struct Biol 2007, 157(1):281-287.
26. Harris L J, Larson S B, Hasel K W, McPherson A: Refined structure of an intact IgG2a monoclonal antibody. Biochemistry 1997, 36(7):1581-1597.
27. Sali A, Blundell T L: Comparative Protein Modeling by Satisfaction of Spatial Restraints. J Mol Biol 1993, 234(3):779-815.
28. Marrink S J, Risselada H J, Yefimov S, Tieleman D P, de Vries A H: The MARTINI force field: Coarse grained model for biomolecular simulations. J Phys Chem B 2007, 111(27):7812-7824.
29. Jo S, Kim T, Iyer V G, Im W: CHARMM-GUI: a web-based graphical user interface for CHARMM. J Comput Chem 2008, 29(11):1859-1865.
30. Periole X, Cavalli M, Marrink S J, Ceruso M A: Combining an Elastic Network With a Coarse-Grained Molecular Force Field: Structure, Dynamics, and Intermolecular Recognition. J Chem Theory Comput 2009, 5(9):2531-2543.
31. Abraham M J, Murtola T, Schulz R, Pall S, Smith J C, Hess B, Lindahl E: GROMACS: High performance molecular simulations through multi-level parallelism from laptops to supercomputers. SoftwareX 2015, 1-2:19-25.
32. Wassenaar T A, Pluhackova K, Bockmann R A, Marrink S J, Tieleman D P: Going Backward: A Flexible Geometric Approach to Reverse Transformation from Coarse Grained to Atomistic Models. J Chem Theory Comput 2014, 10(2):676-690.
33. Schrodinger, LLC: The PyMOL Molecular Graphics System, Version 1.8. In.; 2015.
34. Priceman et al., Front. Immunol., 19 Nov. 2018.

### Embodiments

Embodiment 1. A method of treating cancer in a subject in need thereof, the method comprising administering a therapeutically effective amount of an autologous immune cell bound to a bispecific antibody compound, said bispecific antibody compound comprising an anti-immune cell antibody covalently bound to an anti-cancer antibody through a linker, wherein said linker is attached to a first amino acid within a first hinge region of said anti-immune cell antibody and a second amino acid within a second hinge region of said anti-cancer antibody.

Embodiment 2 The method of embodiment 1, wherein the anti-immune cell antibody is non-covalently bound to the autologous immune cell.

Embodiment 3. The method of one of embodiments 1-2, wherein the anti-immune cell antibody is non-covalently bound to a protein on said autologous immune cell.

Embodiment 4. The method of one of embodiments 1-3, wherein said autologous immune cell is an autologous T cell, autologous NK cell, autologous monocyte, autologous neutrophil, autologous macrophage, or a genetically engineered immune cell.

Embodiment 5. The method of embodiment 4, wherein said genetically engineered immune cell is a CAR T-cell.

Embodiment 6. The method of one of embodiments 1-5, wherein the anti-cancer antibody is non-covalently bound to a cancer protein on a cancer cell, wherein said cancer protein is a carcinoembryonic antigen (CEA), a tumor-associated glycoprotein 72 (TAG-72), an epidermal growth factor receptor (EGFR) protein, CD19, CD20, an epithelial cell adhesion molecule (EpCAM), or a human epidermal growth factor receptor 2 (Her2/neu).

Embodiment 7. The method of one of embodiments 1-6, wherein said cancer is colorectal cancer, ovarian cancer, lung cancer, lymphoma, leukemia, or breast cancer.

Embodiment 8. The method of one of embodiments 1-7, wherein a first Fc domain of said anti-immune cell antibody is oriented in a direction opposite of a second Fc domain of said anti-cancer antibody, wherein said bispecific antibody compound has a 6-lobe morphology.

Embodiment 9. The method of one of embodiments 1-8, wherein the anti-immune cell antibody has a molecular weight of about 150 kDa.

Embodiment 10. The method of one of embodiments 1-9, wherein the anti-cancer antibody has a molecular weight of about 150 kDa.

Embodiment 11. The method of one of embodiments 1-10, wherein the bispecific antibody compound has a molecular weight of about 300 kDa.

Embodiment 12. The method of one of embodiments 1-11, wherein the anti-cancer antibody is covalently attached to a detection moiety.

Embodiment 13. The method of one of embodiments 1-12, wherein the linker has the formula -L¹-L²-L³-L⁴-L⁵-wherein,
L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

Embodiment 14. The method of embodiment 13, wherein L¹ and L⁵ are —CH₂—S—.

Embodiment 15. The method of embodiment 13 or 14, wherein L³ is azocinylene.

Embodiment 16. An autologous immune cell bound to an anti-immune cell antibody, wherein the anti-immune cell antibody comprises a hinge region amino acid, wherein said hinge region amino acid comprises a reactive chemical group side chain.

Embodiment 17. The autologous immune cell of embodiment 16, wherein the reactive chemical group side chain has the formula -L¹-L²-R¹, wherein L¹ and L², are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and R¹ is a reactive functional group.

Embodiment 18. The autologous immune cell of embodiment 17, wherein L¹ is —CH₂—S—.

Embodiment 19. The autologous immune cell of embodiment 17 or 18, wherein the reactive functional group is dibenzocyclooctyne (DBCO).

Embodiment 20. The autologous immune cell of embodiment 17, wherein the reactive functional group is azide.

Embodiment 21. An autologous immune cell bound to a bispecific antibody compound, said bispecific antibody compound comprising an anti-immune cell antibody covalently bound to an anti-cancer antibody through a linker, wherein said linker is attached to a first amino acid within a first hinge region of said anti-immune cell antibody and a second amino acid within a second hinge region of said anti-cancer antibody.

Embodiment 22. The autologous immune cell of embodiment 21, wherein the anti-immune cell antibody is non-covalently bound to the autologous immune cell.

Embodiment 23. The autologous immune cell of embodiment 21 or 22, wherein the anti-immune cell antibody is non-covalently bound to a protein on said autologous immune cell.

Embodiment 24. The autologous immune cell of one of embodiments 21-23, wherein said autologous immune cell is an autologous T cell, autologous NK cell, autologous monocyte, autologous neutrophil, autologous macrophage, or a genetically engineered immune cell.

Embodiment 25. The autologous immune cell of embodiment 24, wherein said genetically engineered immune cell is a CAR T-cell.

Embodiment 26. The autologous immune cell of one of embodiments 21-25, wherein the anti-cancer antibody is non-covalently bound to a cancer protein on a cancer cell, wherein said cancer protein is a carcinoembryonic antigen (CEA), a tumor-associated glycoprotein 72 (TAG-72), an epidermal growth factor receptor (EGFR) protein, CD19, CD20, an epithelial cell adhesion molecule (EpCAM), or a human epidermal growth factor receptor 2 (Her2/neu).

Embodiment 27. The autologous immune cell of one of embodiments 21-26, wherein said cancer is colorectal cancer, ovarian cancer, lung cancer, lymphoma, leukemia, or breast cancer.

Embodiment 28. The autologous immune cell of one of embodiments 21-27, wherein said anti-cancer antibody is covalently bound to a detection moiety.

Embodiment 29. The autologous immune cell of one of embodiments 21-28, wherein a first Fc domain of said anti-immune cell antibody is oriented in a direction opposite of a second Fc domain of said anti-cancer antibody, wherein said bispecific antibody compound has a 6-lobe morphology.

Embodiment 30. The autologous immune cell of one of embodiments 21-29, wherein the anti-immune cell antibody has a molecular weight of about 150 kDa.

Embodiment 31. The autologous immune cell of one of embodiments 21-30, wherein the anti-cancer cell antibody has a molecular weight of about 150 kDa.

Embodiment 32. The autologous immune cell of one of embodiments 21-31, wherein the bispecific antibody compound has a molecular weight of about 300 kDa.

Embodiment 33. The autologous immune cell of one of embodiments 21-32, wherein the linker has the formula -L¹-L²-L³-L⁴-L⁵-, and wherein
L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

Embodiment 34. The autologous immune cell bound to a bispecific antibody compound of embodiment 33, wherein L¹ and L⁵ are —CH₂—S—.

Embodiment 35. The autologous immune cell bound to a bispecific antibody compound of embodiment 33 or 34, wherein L³ is azocinylene.

Embodiment 36. A pharmaceutical composition comprising the autologous immune cell bound to said bispecific antibody of one of embodiments 21-35 and a pharmaceutically acceptable excipient.

Embodiment 37. A process for making an autologous immune cell bound to a bispecific antibody compound, the method comprising:
(a) combining an autologous immune cell and an anti-immune cell antibody in a reaction vessel thereby forming an autologous immune cell-antibody complex, wherein the autologous immune cell-antibody complex comprises said autologous immune cell bound to said anti-immune cell antibody, wherein the anti-immune cell antibody comprises a first hinge region amino acid and said first hinge region amino acid comprises a first reactive chemical group side chain;
(b) combining said autologous immune cell-antibody complex with a second antibody in a reaction vessel thereby forming said autologous immune cell bound to said bispecific antibody compound, wherein:
said second antibody comprises a second hinge region amino acid and said second hinge region amino acid comprises a second reactive chemical group side chain that is reactive with said first reactive chemical group side chain; and
said bispecific antibody compound comprises said anti-immune cell antibody covalently bound to said second antibody through a linker, wherein said linker is attached to said first hinge region amino acid and said second hinge region amino acid.

Embodiment 38. The process of embodiment 37, wherein the first reactive chemical group side chain has the formula -L¹-L²-R¹, wherein
L¹ and L², are independently a bond, —O—, —S—, —C(O)—, —C(O)O, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and
R¹ is a first reactive functional group.

Embodiment 39. The process of embodiment 37 or 38, wherein the second reactive chemical group side chain has the formula -L⁵-L⁴-R², wherein
L⁴ and L⁵, are independently a bond, —O—, —S—, —C(O)—, —C(O)O, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and wherein
R² is a second reactive functional group complementary to said first reactive functional group.

Embodiment 40. The process of embodiment 37, wherein the linker has the formula -L¹-L²-L³-L⁴-L⁵-wherein,
L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

Embodiment 41. The process of one of embodiments 38-40, wherein L¹ and L⁵ are —CH₂—S—.

Embodiment 42. The process of embodiment 40 or 41, wherein L³ is azocinylene.

Embodiment 43. The process of one of embodiments 37-42, wherein the anti-immune cell antibody is non-covalently bound to the autologous immune cell.

Embodiment 44. The process of one of embodiments 37-43, wherein the anti-immune cell antibody is non-covalently bound to a protein on said autologous immune cell.

Embodiment 45. The process of one of embodiments 37-44, wherein said autologous immune cell is an autologous T cell, autologous NK cell, autologous monocyte, autologous neutrophil, autologous macrophage, autologous stem cell, or a genetically engineered immune cell.

Embodiment 46. The process of one of embodiments 37-45, wherein said genetically engineered immune cell is a CAR T-cell.

Embodiment 47. The process of one of embodiments 37-46, wherein the second antibody is an anti-cancer antibody.

Embodiment 48. The process of embodiment 47, wherein said cancer is colorectal cancer, ovarian cancer, lung cancer, lymphoma, leukemia, or breast cancer.

Embodiment 49. The process of one of embodiments 37-48, wherein the second antibody is covalently attached to a detection moiety.

Embodiment 50. The process of one of embodiments 37-49, wherein a first Fc domain of said anti-immune cell antibody is oriented in a direction opposite of a second Fc domain of said second antibody, wherein said bispecific antibody compound has a 6-lobe morphology.

Embodiment 51. The process of one of embodiments 37-50, wherein the anti-immune cell antibody has a molecular weight of about 150 kDa.

Embodiment 52. The process of one of embodiments 37-51, wherein the second antibody has a molecular weight of about 150 kDa.

Embodiment 53. The process of one of embodiments 37-52, wherein the bispecific antibody compound has a molecular weight of about 300 kDa.

What is claimed is:

1. A method of treating cancer in a subject in need thereof, the method comprising administering a therapeutically effective amount of an autologous immune cell bound to a bispecific antibody compound, said bispecific antibody compound comprising an anti-immune cell antibody covalently bound to an anti-cancer antibody through a linker, wherein said linker is attached to a first amino acid within a first hinge region of said anti-immune cell antibody and a second amino acid within a second hinge region of said anti-cancer antibody.

2. The method of claim 1, wherein the anti-immune cell antibody is non-covalently bound to the autologous immune cell.

3. The method of claim 1, wherein the anti-immune cell antibody is non-covalently bound to a protein on said autologous immune cell.

4. The method of claim 1, wherein said autologous immune cell is an autologous T cell, autologous NK cell, autologous monocyte, autologous neutrophil, autologous macrophage, or a genetically engineered immune cell.

5. The method of claim 4, wherein said genetically engineered immune cell is a CAR T-cell.

6. The method of claim 1, wherein the anti-cancer antibody is non-covalently bound to a cancer protein on a cancer cell, wherein said cancer protein is a carcinoembryonic antigen (CEA), a tumor-associated glycoprotein 72 (TAG-72), an epidermal growth factor receptor (EGFR) protein, CD19, CD20, an epithelial cell adhesion molecule (EpCAM), or a human epidermal growth factor receptor 2 (Her2/neu).

7. The method of claim 1, wherein said cancer is colorectal cancer, ovarian cancer, lung cancer, lymphoma, leukemia, or breast cancer.

8. The method of claim 1, wherein a first Fc domain of said anti-immune cell antibody is oriented in a direction opposite of a second Fc domain of said anti-cancer antibody, wherein said bispecific antibody compound has a 6-lobe morphology.

9. The method of claim 1, wherein the anti-immune cell antibody has a molecular weight of about 150 kDa.

10. The method of claim 1, wherein the anti-cancer antibody has a molecular weight of about 150 kDa.

11. The method of claim 1, wherein the bispecific antibody compound has a molecular weight of about 300 kDa.

12. The method claim 1, wherein the anti-cancer antibody is covalently attached to a detection moiety.

13. The method of claim 1, wherein the linker has the formula -L¹-L²-L³-L⁴-L⁵-wherein,
L¹, L², L¹, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

14. The method of claim 13, wherein L¹ and L⁵ are —CH₂—S—.

15. The method of claim 13, wherein L³ is azocinylene.

16. An autologous immune cell bound to an anti-immune cell antibody, wherein the anti-immune cell antibody comprises a hinge region amino acid, wherein said hinge region amino acid comprises a reactive chemical group side chain.

17. The autologous immune cell of claim 16, wherein the reactive chemical group side chain has the formula -L¹-L²-R¹, wherein
L¹ and L², are independently a bond, —O—, —S—, —C(O)—,
—C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and
R¹ is a reactive functional group.

18. The autologous immune cell of claim 17, wherein L¹ is —CH₂—S—.

19. The autologous immune cell of claim 17, wherein the reactive functional group is dibenzocyclooctyne (DBCO).

20. The autologous immune cell of claim 17, wherein the reactive functional group is azide.

21. An autologous immune cell bound to a bispecific antibody compound, said bispecific antibody compound comprising an anti-immune cell antibody covalently bound to an anti-cancer antibody through a linker, wherein said linker is attached to a first amino acid within a first hinge region of said anti-immune cell antibody and a second amino acid within a second hinge region of said anti-cancer antibody.

22. The autologous immune cell of claim 21, wherein the anti-immune cell antibody is non-covalently bound to the autologous immune cell.

23. The autologous immune cell of claim 21, wherein the anti-immune cell antibody is non-covalently bound to a protein on said autologous immune cell.

24. The autologous immune cell of claim 21, wherein said autologous immune cell is an autologous T cell, autologous NK cell, autologous monocyte, autologous neutrophil, autologous macrophage, or a genetically engineered immune cell.

25. The autologous immune cell of claim 24, wherein said genetically engineered immune cell is a CAR T-cell.

26. The autologous immune cell of claim 21, wherein the anti-cancer antibody is non-covalently bound to a cancer protein on a cancer cell, wherein said cancer protein is a carcinoembryonic antigen (CEA), a tumor-associated glycoprotein 72 (TAG-72), an epidermal growth factor receptor (EGFR) protein, CD19, CD20, an epithelial cell adhesion molecule (EpCAM), or a human epidermal growth factor receptor 2 (Her2/neu).

27. The autologous immune cell of claim 21, wherein said cancer is colorectal cancer, ovarian cancer, lung cancer, lymphoma, leukemia, or breast cancer.

28. The autologous immune cell of claim 21, wherein said anti-cancer antibody is covalently bound to a detection moiety.

29. The autologous immune cell of claim 21, wherein a first Fc domain of said anti-immune cell antibody is oriented in a direction opposite of a second Fc domain of said anti-cancer antibody, wherein said bispecific antibody compound has a 6-lobe morphology.

30. The autologous immune cell of claim 21, wherein the anti-immune cell antibody has a molecular weight of about 150 kDa.

31. The autologous immune cell of claim 21, wherein the anti-cancer cell antibody has a molecular weight of about 150 kDa.

32. The autologous immune cell claim 21, wherein the bispecific antibody compound has a molecular weight of about 300 kDa.

33. The autologous immune cell of one of claim 21, wherein the linker has the formula -L¹-L²-L³-L⁴-L⁵-, and wherein
L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

34. The autologous immune cell bound to a bispecific antibody compound of claim 33, wherein L¹ and L⁵ are —CH₂—S—.

35. The autologous immune cell bound to a bispecific antibody compound of claim 33, wherein L³ is azocinylene.

36. A pharmaceutical composition comprising the autologous immune cell bound to said bispecific antibody of claim 21 and a pharmaceutically acceptable excipient.

37. A process for making an autologous immune cell bound to a bispecific antibody compound, the method comprising:
(a) combining an autologous immune cell and an anti-immune cell antibody in a reaction vessel thereby forming an autologous immune cell-antibody complex, wherein the autologous immune cell-antibody complex comprises said autologous immune cell bound to said anti-immune cell antibody, wherein the anti-immune cell antibody comprises a first hinge region amino acid and said first hinge region amino acid comprises a first reactive chemical group side chain;
(b) combining said autologous immune cell-antibody complex with a second antibody in a reaction vessel thereby forming said autologous immune cell bound to said bispecific antibody compound, wherein:
said second antibody comprises a second hinge region amino acid and said second hinge region amino acid comprises a second reactive chemical group side chain that is reactive with said first reactive chemical group side chain; and
said bispecific antibody compound comprises said anti-immune cell antibody covalently bound to said second antibody through a linker, wherein said linker is attached to said first hinge region amino acid and said second hinge region amino acid.

38. The process of claim 37, wherein the first reactive chemical group side chain has the formula -L¹-L²-R¹, wherein
L¹ and L², are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and
R¹ is a first reactive functional group.

39. The process of claim 37, wherein the second reactive chemical group side chain has the formula -L⁵-L⁴-R², wherein
L⁴ and L⁵, are independently a bond, —O—, —S—, —C(O)—, —C(O)O, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, and wherein
R² is a second reactive functional group complementary to said first reactive functional group.

40. The process of claim 37, wherein the linker has the formula -L¹-L²-L³-L⁴-L⁵-wherein,
L¹, L², L³, L⁴, and L⁵ are independently a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

41. The process of claim 38, wherein L¹ and L⁵ are —CH₂—S—.

42. The process of claim 40, wherein L³ is azocinylene.

43. The process of claim 37, wherein the anti-immune cell antibody is non-covalently bound to the autologous immune cell.

44. The process of claim 37, wherein the anti-immune cell antibody is non-covalently bound to a protein on said autologous immune cell.

45. The process of claim 37, wherein said autologous immune cell is an autologous T cell, autologous NK cell, autologous monocyte, autologous neutrophil, autologous macrophage, autologous stem cell, or a genetically engineered immune cell.

46. The process of claim 37, wherein said genetically engineered immune cell is a CAR T-cell.

47. The process of claim 37, wherein the second antibody is an anti-cancer antibody.

48. The process of claim 47, wherein said cancer is colorectal cancer, ovarian cancer, lung cancer, lymphoma, leukemia, or breast cancer.

49. The process of claim 37, wherein the second antibody is covalently attached to a detection moiety.

50. The process of claim 37, wherein a first Fc domain of said anti-immune cell antibody is oriented in a direction opposite of a second Fc domain of said second antibody, wherein said bispecific antibody compound has a 6-lobe morphology.

51. The process of claim 37, wherein the anti-immune cell antibody has a molecular weight of about 150 kDa.

52. The process of claim 37, wherein the second antibody has a molecular weight of about 150 kDa.

53. The process of claim 37, wherein the bispecific antibody compound has a molecular weight of about 300 kDa.
